(19) United States
(12) Patent Application Publication
(10) Pub. No.: US 2023/0340452 A1
(43) Pub. Date: Oct. 26, 2023

(54) METHODS OF CAPTURING, SEPARATING, AND/OR ENRICHING LOW ABUNDANT TARGET BIOMOLECULES

(71) Applicant: The Governors of the University of Alberta, Edmonton (CA)

(72) Inventors: Weiwei WANG, Edmonton (CA); Wei-Zheng SHEN, Edmonton (CA); Fereshte DAMAVANDI, Edmonton (CA); Carlo MONTEMAGNO, Edmonton (CA); Gane Ka-Shu WONG, Edmonton (CA)

(21) Appl. No.: 17/793,164

(22) PCT Filed: Jan. 15, 2021
(86) PCT No.: PCT/CA2021/050043

§ 371 Date: Jul. 15, 2022

Related U.S. Application Data
(60) Provisional application No. 62/962,111, filed on Jan. 16, 2020.

(51) Int. Cl. C12N 15/10; C12Q 1/70

(52) U.S. Cl. CPC C12N 15/1013; C12Q 1/707; C12Q 2600/118

(57) ABSTRACT

Provided are methods for capturing one or more target biomolecules in a sample and/or enriching one or more target biomolecules in a sample. Also provided are methods for diagnosis and/or prognosis of a disease and/or disorder associated using the methods and capture compounds.

### CROSS-REFERENCE TO RELATED APPLICATIONS

This application claims priority to and benefit of U.S. Provisional Patent Application Ser. No. 62/962,111 filed on Jan. 16, 2020, which is hereby incorporated by reference in its entirety.

### TECHNICAL FIELD

The present disclosure generally relates to capture compounds and methods and uses thereof. In particular, the present disclosure relates to methods for capturing, separating, and/or enriching a low abundant target biomolecule from a sample, such as for example a target nucleic acid from a biological sample.

### BACKGROUND

Detection of target biomolecules (e.g. nucleic acids) is an important step in a number of biological probe based assays, including next-generation sequencing, amplification, and clinical diagnosis.¹⁻³ As well, gene panels use nucleic acid detection to identify the presence/abundance of target nucleic acids, such as mutated or non-mutated genes, that may be indicative of a genetic diagnosis. However, detection of low abundant target biomolecules (e.g. DNA/RNA) is challenging because the target sequences are often buried under an enormous background of human genomic and/or non-human metagenomic sequences.

Detection systems have been developed using designed oligonucleotides as probes to capture the target nucleic acids.⁴ Probe-based DNA/RNA capture techniques can be divided into two main categories, namely fluid-phase and solid-phase. Existing fluid-phase methods suffer from drawbacks such as solvent toxicity and being time consuming processes. Thus, solid-phase techniques are generally viewed as a more reliable capture strategy.⁵

In solid-phase probe capture techniques, the solid support surface properties play a key role in controlling physiochemical interactions.⁶⁻⁷ Nano solid-supports have been implemented in nucleic acid detection systems.⁷⁻⁸ Iron oxide nanoparticles are an example of nano solid-supports that can enable easier separation and quicker process times owing to their magnetic properties.⁹⁻¹⁷ However, current bead-based capture systems exhibit a limited recovery rate for low abundance target sequences. Capture assays based on commercial streptavidin coupled magnetic beads¹² remain surprisingly inefficient because non-specific binding of background nucleic acids (e.g. human genomic sequence) to the surface of the bead can potentially overwhelm any nucleic acid that is intended to be captured from the sample.

Attempts to improve target nucleic acid capture efficiency by modifying the magnetic surface of a solid-phase probe have been made, such as modification of the magnetic nanoparticles' surface using low-fouling oligo ethylene glycol methacrylate to decrease non-specific binding.¹⁸ However, limited recovery rates, as well as non-specificity in respect of targets, persists because of low nucleic acid binding efficiency and high levels of non-specific interaction.¹⁹⁻²³

Next generation sequencing (NGS) can be effective for surveying medium-to-high abundance targets, but is more problematic for low abundance targets, such as for example viruses and bacteria at the initial pre-symptomatic stage or the later chronic stage of infection, non-abundant viruses and bacteria in metagenomics samples, or subsets of gene transcripts that are expressed at low levels.

A need therefore exists to develop improved methods for capturing, separating, and/or enriching low abundant target biomolecules in a sample, such as a biological sample.

### SUMMARY

The present disclosure provides methods for capturing and/or enriching a low abundant target biomolecule, such as a low abundant target nucleic acid, from a sample.

An advantage of the present disclosure is the provision of methods having improved characteristics over existing technologies, such as for example and without limitation, increased binding efficiency, improved sensitivity and improved specificity.

In an embodiment, the present disclosure relates to a method for capturing a low abundant target biomolecule, the method comprising: providing a capture compound comprising a silica-coated nanoparticle conjugated to one or more biomolecule probes; and incubating the capture compound with a sample comprising the low abundant target biomolecule to capture the low abundant target biomolecule.

In an embodiment, the present disclosure relates to a method of enriching a low abundant target biomolecule in a biological sample, comprising: providing an unbound capture compound comprising a silica-coated nanoparticle conjugated to one or more biomolecule probes; incubating the capture compound with a biological sample comprising the low abundant target biomolecule; and performing an amplification reaction to enrich the low abundant target biomolecule in the biological sample. In an embodiment, the amplification reaction is a polymerase chain reaction (PCR).

In an embodiment, the present disclosure relates to a method for diagnosis of a disease and/or disorder associated with an infectious agent or a mutated or non-mutated nucleotide sequence of a nucleic acid of a human genome, the method comprising: providing a biological sample from a subject; and capturing a low abundant target biomolecule of an infectious agent and/or a low abundant target nucleic acid which comprises a mutated or non-mutated nucleotide sequence of a nucleic acid of a human genome, by using a capture compound comprising a silica-coated nanoparticle conjugated to one or more biomolecule probes in a capture assay.

In an embodiment, the present disclosure relates to a method for prognosis of a disease and/or disorder associated with an infectious agent or a mutated or non-mutated nucleotide sequence of a nucleic acid of a human genome, the method comprising: providing a biological sample from a subject; capturing a low abundant target biomolecule of an infectious agent and/or a low abundant target nucleic acid which comprises a mutated or non-mutated nucleotide sequence of a nucleic acid of a human genome, using a capture compound comprising a silica-coated nanoparticle conjugated to one or more biomolecule probes in a capture assay; and detecting for a quantity of the low abundant target biomolecule and/or the low abundant target nucleic acid, wherein either: an elevated or reduced level of the low abundant target biomolecule and/or the low abundant target nucleic acid in the biological sample as compared to a predefined value is indicative of a poor prognosis or active disease state; or an elevated or reduced level of the low abundant target biomolecule and/or the low abundant target nucleic acid in the biological sample as compared to an earlier sample from the subject is indicative of a poor prognosis or active disease state.

In an embodiment, the present disclosure relates to the use of a capture compound comprising a silica-coated nanoparticle conjugated to one or more biomolecule probes for improving capture specificity of one or more low abundant target nucleic acids of a gene panel.

In an embodiment, the present disclosure relates to the use of a capture compound comprising a silica-coated nanoparticle conjugated to one or more biomolecule probes for capturing a low abundant target biomolecule in a biological sample.

In an embodiment, the present disclosure relates to the use of a capture compound comprising a silica-coated nanoparticle conjugated to one or more biomolecule probes for enrichment of a low abundant target biomolecule in a biological sample by a downstream amplification assay.

In an embodiment, the present disclosure relates to a capture assay kit for detection of a low abundant target biomolecule, the capture assay kit comprising: a capture compound comprising a silica-coated nanoparticle conjugated to one or more biomolecule probes and one or more reagents for low abundant capture.

Other aspects and features of the capture compounds and methods of the present disclosure will become apparent to those ordinarily skilled in the art upon review of the following description of specific embodiments. Without being bound by any particular theory, the embodiments of the present disclosure may improve the ability to capture a target nucleic acid from a sample.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a scanning electron microscopy (SEM) image of an embodiment of iron oxide nanoparticles of the present disclosure (FIG. 1A) and a histogram of iron oxide nanoparticles size; size distribution computed by Image J software (FIG. 1B).

FIG. 2 shows a scanning electron microscopy image (FIG. 2A) and a high-resolution transmission electron microscopy image (FIG. 2B) of the core-shell structure of an embodiment of a silica-coated iron oxide nanoparticle of the present disclosure.

FIG. 3 shows saturation curves for probe immobilization as a function of the amount of DNA in the reaction. FIG. 3A shows the curve for probe C and FIG. 3B shows the curve for probe F, both normalized by the amount of nanoparticles. iDNA refers to DNA immobilized on the beads.

FIG. 4 illustrates the influence of reaction time on probe immobilization. FIG. 4A is for probe C and FIG. 4B is for probe F. iDNA refers to DNA immobilized on the beads. iDNA/nanoparticle ratio was set to 2.4 μM.

FIG. 5 illustrates the influence of copper concentration on probe immobilization. FIG. 5A is for probe C and FIG. 5B is for probe F. iDNA refers to DNA immobilized on the beads. iDNA/nanoparticle ratio was set to 2.4 μM.

FIG. 6 shows an illustration of an exemplary nanoparticle capture method of the present disclosure. FIG. 6A shows DNA-conjugated iron oxide silica-coated nanoparticles incubated in the target solution to promote hybridization. Non-specifically hybridized DNA/RNA is removed by washes. Specifically hybridized DNA/RNA is retained on the magnetically immobilized beads. FIG. 6B shows preparation of the iron oxide silica-coated nanoparticles by: i) iron oxide nanoparticle synthesis, ii) silica coating, iii) azide functionalization, and iv) conjugation with DNA probes through click chemistry.

FIG. 7 shows confirmation of probe-target hybridization using an exemplary DNA probe modified silica-coated iron oxide nanoparticle of the present disclosure. FIG. 7A is a bright field microscopy image, FIG. 7B is an image under an Alex Fluor 488 (green) filter showing nanoparticles conjugated to Alexa Fluor 488 (green) labeled DNA probes, and FIG. 7C is an image under a Cy5 (red) filter showing nanoparticles conjugated to Cy55 (red) labeled complementary DNA.

FIG. 8 shows a non-limiting example of the capture specificity of a method of the present disclosure using synthetic DNA gblocks (A and B) representing two distinct regions of the HCV genome in a simulated clinical sample background. Copy number was measured by real-time qPCR, for gblocks A and B as well as for a human housekeeping gene B2M, both before (FIG. 8A) and after (FIG. 8B) capture. FIG. 8C shows enrichment factor as a ratio of copy numbers before and after capture.

FIG. 9 shows a non-limiting example of the capture of eight target regions of the HCV genome (labels A to H) using a method of the present disclosure. FIG. 9A shows copy numbers at different hybridization times. FIG. 9B shows that at 24 h, the DNA probe-clicked silica-coated iron oxide nanoparticles of the present disclosure outperformed streptavidin in all regions, and at 4 h in most regions.

FIG. 10 shows a non-limiting example of the performance of the DNA probe-clicked silica-coated iron oxide nanoparticles of the present disclosure with plasma of HCV-infected patients. FIG. 10A shows copy numbers from a fast (4 h hybridization) protocol, demonstrating consistently stable signals from all eight HCV targets fragments. FIG. 10B shows enrichment factor as a function of sample (main plot) and viral titer (inset).

### DETAILED DESCRIPTION

Unless otherwise defined, all technical and scientific terms used herein generally have the same meaning as commonly understood by one of ordinary skill in the art to which this disclosure pertains. Exemplary terms are defined below for ease in understanding the subject matter of the present disclosure.

### Definitions

The term “a” or “an” refers to one or more of that entity; for example, “a functionalized oligonucleotide probe” refers to one or more functionalized oligonucleotide probes or at least one functionalized oligonucleotide probe. As such, the terms “a” (or “an”), “one or more” and “at least one” are used interchangeably herein. In addition, reference to an element or feature by the indefinite article “a” or “an” does not exclude the possibility that more than one of the elements or features are present, unless the context clearly requires that there is one and only one of the elements.

“About”, when referring to a measurable value such an amount of a compound or agent, dose, time, temperature, and the like, is meant to encompass variations of ±20%, ±10%, ±5%, ±1%, ±0.5% or ±0.1% of the specified amount. When the value is a whole number, the term about is meant to encompass decimal values, as well the degree of variation just described. It is to be understood that such a variation is always included in any given value provided herein, whether or not it is specifically referred to.

“And/or” refers to and encompasses any and all possible combinations of one or more of the associated listed items (e.g. one or the other, or both), as well as the lack of combinations when interrupted in the alternative (or).

“Comprise” as is used in this description and in the claims, and its conjugations, is used in its non-limiting sense to mean that items following the word are included, but items not specifically mentioned are not excluded.

### Methods of Capturing a Low Abundant Target Biomolecule

In many samples analyzed for clinical and/or research purposes, the biomolecules of interest are typically found in mixtures of asymmetrical abundance, with low abundant targets hidden under a background of human genomic and/or non-human metagenomic sequences. While approaches have been developed for target enrichment, many technologies that combine hybridization capture and next generation sequencing are onerous, expensive, and time-consuming. Further, current bead-based capture systems exhibit a limited recovery rate for low abundant target sequences.

The present disclosure provides improved methods for capturing, separating, and/or enriching a low abundant target biomolecule from a sample.

Without being bound by a particular theory, some advantages of the methods and capture compounds disclosed herein include improved sensitivity and specificity for a low abundant target biomolecule and/or a reduction in non-specific binding. As used herein, the term “improved efficiency” may be in reference to any one or more of these properties, as context dictates.

For example, the methods disclosed herein are particularly advantageous in that the improvement in specificity for low abundant target nucleic acids significantly reduces the cost of downstream sequencing. By having improved target specificity, there is a significant reduction in non-target capture which, in turn, significantly reduces undesirable sequencing of extraneous or non-target DNA. This is a major advantage of the methods disclosed herein.

As used herein, the term “sensitivity” refers to a measure of how strong or at what abundance a stimulus has to be before a method, system or assay can react or respond to it. The lower the strength or abundance of the stimulus that is still sufficient to elicit a reaction or response, the higher the sensitivity. In an embodiment, abundance may be in reference to the relative proportion (e.g. copy number, w/w percentage, or concentration) of the stimulus in relation to non-target or background components. In the context of the present disclosure, “sensitivity” refers to the ability of capture compounds in the methods herein to react or respond to (e.g. capture and/or enrich) the target biomolecule at levels of low abundance. In embodiments herein, the methods of the present disclosure are capable of improved sensitivity, for example in comparison to other methods that do not employ a capture compound or method steps as disclosed herein.

As used herein, the term “specificity” refers to a measure of the ability of a method, system or assay to react or respond only to the correct target stimulus in a sample. For example, specificity reflects the ability to react or respond only to the correct target stimulus in the presence of interference or background in the sample. The greater the ability to react or respond to the correct target stimulus and not with other components in the sample, the greater the specificity. In the context of the present disclosure, “specificity” refers to the ability of capture compounds in the methods herein to react or respond to (e.g. capture and/or enrich) only the correct target biomolecule and not other components of the sample. The other components in the sample may include for example and without limitation non-target nucleic acids (e.g. DNA/RNA), proteins, enzymes, and other biological materials. In embodiments herein, the methods of the present disclosure are capable of improved specificity, for example in comparison to other methods that do not employ a capture compound or method steps as disclosed herein.

Herein, the terms “sensitivity” and “specificity” are used in a context compatible with their usage in the medical (and statistical) fields where both are continuous variables. This is in contrast to how these terms are typically used in the analytic chemistry field, where “specificity” is often treated as an absolute (i.e. an assay is either specific or not). As such, in the analytic chemistry field, the term “selectivity” was coined to serve a similar purpose to allow for varying degrees of performance.²⁴ As used herein, the term “specificity” is compatible, or interchangeable, with the term “selectivity” as defined by the analytic chemistry community.

As used herein, the term “non-specific binding” is intended to refer to binding to something other than is desired or intended, e.g. other than the low abundant target. Without being bound by a particular theory, non-specific binding may lead to dominant signals from non-target molecules, which in turn may lead to inefficient downstream processes. Therefore, a reduction in non-specific binding is generally desirable. Non-limiting examples of downstream processes include real-time polymerase chain reaction (PCR), loop-mediated isothermal amplification (LAMP), quantitative PCR (qPCR), or sequencing methods, for example, Sanger sequencing, next-generation sequencing, or nanopore sequencing.

In an embodiment, the present disclosure provides a method for capturing a low abundant target biomolecule, the method comprising: providing a capture compound comprising a silica-coated nanoparticle conjugated to one or more biomolecule probes; and incubating the capture compound with a sample comprising the low abundant target biomolecule to capture the low abundant target biomolecule.

As used herein, the term “low abundant target” is meant to refer to a target having a substantially lower copy number or concentration as compared to the non-target material. By “non-target material”, it is meant anything in the sample other than the intended target biomolecule. In an embodiment, the non-target material is of a similar chemical nature as the target biomolecule (e.g. nucleic acids or proteins). In an embodiment, the non-target material is genomic background.

In an embodiment, the low abundant target biomolecule is a nucleic acid and is present in a sample at a lower copy number than the limit of detection of a sequencing and/or amplification method; for example, a quantitative polymerase chain reaction (qPCR). In an embodiment, the low abundant target nucleic acid is present in a sample at a copy number of less than 10⁸ copies/100 ng of nucleic acid (DNA and/or RNA) in the sample. More particularly, in an embodiment, the low abundant target nucleic acid is present in a sample at a copy number of less than 10⁷ copies, 10⁶ copies, 10⁵ copies, 10⁴ copies, 10³ copies, or less, per 100 ng of nucleic acid in the sample. In an embodiment, the low abundant target nucleic acid is present in a sample at a copy number of between about 10² and about 10⁶ copies/100 ng of nucleic acid in the sample, more particularly between about 10³ and about 10⁴ copies/100 ng of nucleic acid in the sample.

In an embodiment, the low abundant target biomolecule is less than 10%, less than 9%, less than 8%, less than 7%, less than 6%, less than 5%, less than 4%, less than 3%, less than 2%, or less than 1% of the total non-target background (e.g. genomic background, protein background, cellular component background, and/or non-human metagenomics background) in the sample.

In an embodiment, the low abundant target biomolecule (e.g. a target nucleic acid) and is less than 5% of the total genomic background in a biological sample. In an embodiment, the low abundant target biomolecule is less than 1% of the total genomic background in a biological sample. In an embodiment, the low abundant target biomolecule is less than 0.1% of the total genomic background in the biological sample. In an embodiment, the low abundant target biomolecule is less than 0.01% of the total genomic background in the biological sample. In an embodiment, the low abundant target biomolecule is less than 0.001% of the total genomic background in the biological sample. In an embodiment, the low abundant target biomolecule is a nucleic acid.

As used herein, the term “biomolecule” is meant to refer to a biological molecule that is produced by cells and living organisms, or synthetic counterparts thereof which may be subject to any number of modifications. In an embodiment, the biomolecule may be any compound, molecule, or particle of interest in the sample. Exemplary embodiments include, without limitation, nucleic acids, proteins, enzymes, carbohydrates, lipids, and nutrients. In an embodiment, the low abundant target biomolecule is a nucleic acid or a protein. In a particular embodiment, the low abundant target biomolecule is a target nucleic acid, such as a DNA (e.g human autosomal or mitochondrial; bacterial, viral, etc.), RNA (e.g. mRNA, tRNA, rRNA) or mixed DNA/RNA nucleic acid.

In an embodiment, the low abundant target biomolecule may be a biomolecule of an infectious agent. The infectious agent may for example and without limitation be a virus, bacteria, or fungus. Thus, in an embodiment, the low abundant target biomolecule may be a nucleic acid of a viral genome, a bacterial genome, or a fungal genome. The genome of the infectious agent may be DNA or RNA.

Exemplary, and non-limiting, viruses include Cowpoxvirus, Vaccinia virus, Pseudocowpox virus, Human herpesvirus 1, Human herpesvirus 2, Cytomegalovirus, Human adenovirus A-F, Polyomavirus, Human papillomavirus (HPV), Parvovirus, Hepatitis A virus, Hepatitis B virus, Hepatitis C virus (HCV), Human immunodeficiency virus, Orthoreovirus, Rotavirus, Ebola virus, parainfluenza virus, influenza A virus, influenza B virus, influenza C virus, Measles virus, Mumps virus, Rubella virus, Pneumovirus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus or varicella. In a particular embodiment, the virus is Hepatitis C virus. In a particular embodiment, the virus is an influenza virus. In a particular embodiment, the virus is a Coronavirus.

Exemplary, and non-limiting, bacteria include Acinetobacter baumannii, Anthrax (Bacillus anthracis), Brucella, Bordetella pertussis, Burkholderia Cepacia, Camplobacter, Candida, Chlamydia pneumoniae, Chlamydia psittaci, Cholera, Clostridium botulinum, Clostridium Difficile, Clostridium perfringens, Coccidioides immitis, Cryptococcus, Diphtheria, Escherichia coli, Haemophilus influenzae, Helicobacter pylori, Klebsiella pneumonia, Legionella, Leptospira, Listeria, Meningococcus, Mycoplasma pneumoniae, Mycobacterium, Mycobacterium Tuberculosis, Neisseria gonorrhoeae, Pertussis, Pneumonia, Pseudomonas Aeruginosa, Salmonella, Shigella, Staphylococcus, Staphylococcus Aureus, Streptococcus aureaus, Streptococcus pneumonia, Streptococcus pyogenes and Yersinia enterocolitica.

In another embodiment, the low abundant target biomolecule may be an expression product of an infectious agent, such as a messenger RNA (mRNA). In an embodiment, the low abundant target biomolecule may be a peptide, such as a human peptide (modified or unmodified) or a peptide of a viral proteome, a bacterial proteome, or a fungal proteome. As used herein, a “peptide” is a polynucleotide of any length (e.g. a protein).

As used herein, the term “target nucleic acid” refers to a nucleic acid of interest for capture and/or separation from a sample. By “nucleic acid” it is meant to refer to a chain of at least two or more nucleotides. As the skilled person will appreciate, nucleic acids are complex organic substances present in living things (e.g. DNA or RNA), whose molecules typically consist of many nucleotides linked in a long chain. With respect to the present disclosure, the low abundant target nucleic acid may be of any length or any sequence, and may be naturally occurring or non-naturally occurring. In an embodiment and without limitation, the target nucleic acid may be, or be part of, a gene, a gene fragment, an exon, an intron, an mRNA, a tRNA, rRNA or snRNA.

In an embodiment, the low abundant target nucleic acid may be a nucleic acid present within a human subject. For example and without limitation, the target nucleic may comprise a mutated or non-mutated nucleotide sequence of a nucleic acid of a human genome. As used herein, the term “mutated nucleotide sequence” is intended to refer to a nucleotide sequence that comprises a genetic alteration in the nucleotide sequence. The mutated nucleotide sequence may comprise one or more nucleotide substitutions, additions and/or deletions as compared to the non-mutated counterpart. The mutated nucleotide sequence may be within an intron or exon. In an embodiment, the mutated nucleotide sequence is within a nucleotide sequence that makes up a gene. In an embodiment, the gene is associated with a disease or disorder. In an embodiment, the gene is associated with cancer.

In an embodiment, the low abundant target biomolecule (e.g. nucleic acid) may be from a fetus. For example, the biological sample may be a maternal blood sample in which the low abundant target biomolecule is present. Thus, the methods disclosed herein may be advantageous in respect of non-invasive fetal diagnostics.

As used herein, the term “non-mutated nucleotide sequence” is intended to refer to any sequence of the human genome, including any genetic polymorphisms that exist between individuals, groups, or populations. As such, reference to a non-mutated nucleotide sequence encompasses naturally occurring polymorphisms. The non-mutated nucleotide sequence may be within an intron or exon. In an embodiment, the non-mutated nucleotide sequence is within a nucleotide sequence that makes up a gene. In an embodiment, altered expression of the gene may be associated with a disease or disorder.

In an embodiment, the low abundant target nucleic acid may be a synthetic oligonucleotide. As used herein, the term “synthetic oligonucleotide” is meant to refer to an oligonucleotide prepared by a chemical reaction, for example in a laboratory setting or by an oligonucleotide synthesizer instrument. Synthetic nucleotides are not limited to synthesizing DNA and RNA only in a 5′ to 3′ direction. Synthetic oligonucleotides may comprise a sequence existing in nature, or not.

In an embodiment, the synthetic oligonucleotide may be a linker. By “linker”, it is meant to refer to an oligonucleotide that is itself capable of binding a target as described herein. Thus, in an embodiment, the capture compounds of the present disclosure may capture a low abundant target nucleotide via interaction with a linker. In such embodiments, the biomolecule probe of the capture compound is an oligonucleotide probe that targets the linker, rather than the target oligonucleotide itself. In alternate embodiments, the linker may be a compound other than a synthetic oligonucleotide, such as for example a peptide or protein.

In an embodiment, the step of providing the capture compound comprising a silica-coated nanoparticle conjugated to one or more biomolecule probes comprises synthesizing the capture compound, such as for example by means disclosed herein. In an embodiment, the step of providing comprises adding the capture compound to the sample. The capture compound may be added to the sample with agitation, mixing or any other means to disperse the capture compound throughout the sample. The providing may also comprise adding the sample to a solution in which the capture compound is present or combining the capture compound with the sample in another reagent or solution.

As used herein, the term “capture compound” refers to a composition of matter that is capable of binding to a low abundant target such that the low abundant target can be captured from within a sample, separated from a sample and/or enriched. By capturing and/or separating the low abundant target biomolecule, sequencing may be performed to provide various types of information, such as for example and without limitation the abundance of the target, the species of an infectious agent and/or whether it is a drug-resistant variant, and the presence and/or absence of a genetic variant.

The capture compounds disclosed herein may be used for capturing and/or enriching a low abundant target biomolecule, such as a low abundant target nucleic acid, in a sample. In an embodiment the sample is a biological sample, such as for example and without limitation blood, urine, tissue, or saliva. In other embodiments, the sample is an environmental sample (e.g. water sample) or a sewage sample.

In some embodiments of the methods herein, the capture compound may be an unbound capture compound. By “unbound capture compound”, it is meant that the capture compound is not immobilized, affixed or otherwise bound to a substrate or surface. Rather, the capture compound is free in the sample. For example, and without limitation, unbound capture compounds are not immobilized to the surface of a plate or slide. Without being bound to any particular theory, it is believed that unbound capture compounds are advantageous in capturing and/or enriching low abundant target biomolecules.

The capture compound of the present disclosure comprises a silica-coated nanoparticle. Suitable nanoparticles include, but are not limited to metallic (e.g., gold, silver, copper), semiconducting (e.g. CdSe, CdS), or magnetic (e.g. iron oxide) nanoparticles. In a particular embodiment, the nanoparticle used in the capture compounds disclosed herein is advantageously a magnetic nanoparticle. An advantage of magnetic nanoparticles is that they can be separated from a mixture by magnetic attraction.

In an embodiment, the nanoparticles have a substantially spherical shape. In an embodiment, the diameter of an individual nanoparticle is between about 10 nm and about 500 nm. In an embodiment, the diameter of the individual nanoparticle is between about 200 nm and about 275 nm, more particularly between about 220 nm and about 260 nm. In an embodiment, the diameter of the individual nanoparticle is about 220 nm, about 225 nm, about 230 nm, about 235 nm, about 240 nm, about 245 nm, about 250 nm, about 255 nm or about 260 nm. In a particular embodiment, the diameter of the individual nanoparticle is about 240 nm. In an embodiment, the nanoparticles have a non-spherical shape including, for example, a rod shape. The nanoparticle may be synthesized by any method known in the art or may be commercially sourced.

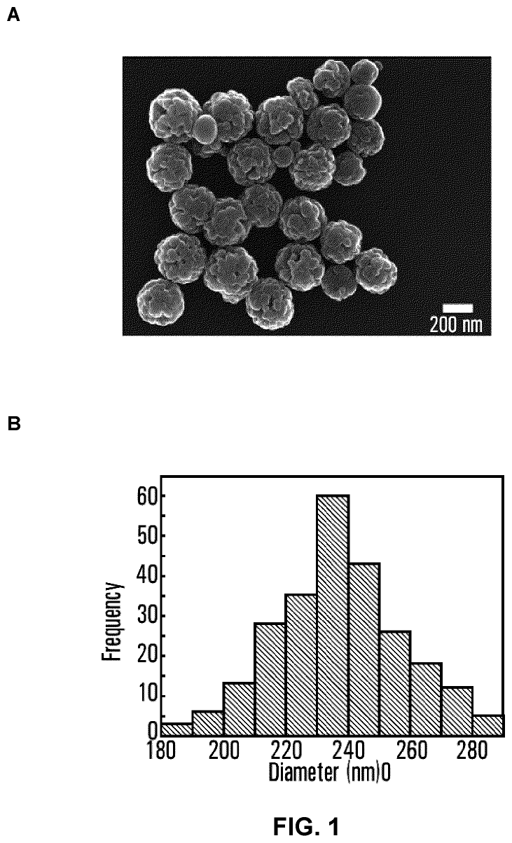

In an embodiment, the nanoparticle is an iron oxide nanoparticle. In an embodiment, the iron oxide nanoparticle is prepared by dissolving FeCl₃ in ethylene glycol, adding sodium acetate, and heating in an autoclave. The skilled person will appreciate that other methods of preparing the iron oxide nanoparticles may be used. In an embodiment, the iron oxide nanoparticles are of a substantially spherical shape. In an embodiment, the iron oxide nanoparticles have a diameter of about 240 nm. FIG. 1A shows a scanning electron microscopy (SEM) image and FIG. 1B shows a histogram of iron oxide nanoparticles prepared as described above and in Example 1.

As used herein, the term “silica-coated” can be used interchangeably with “silica shell” and is intended to refer to a silica coating covering at least a portion of the surface of the nanoparticle. The silica coating may provide uniform coverage of the nanoparticle surface, or not. In an embodiment, substantially all of the nanoparticle is covered by the silica coating. In an embodiment, all of the nanoparticle is covered by the silica coating.

In an embodiment, the silica coating of the silica-coated nanoparticle is provided by a hydrolysis-condensation reaction between the nanoparticle and tetraethyl orthosilicate in a sol-gel process. However, the skilled person will appreciate that other coating methods may be used to provide the silica coating. In an embodiment, the silica coating of the silica-coated iron oxide nanoparticle is provided by the hydrolysis-condensation reaction between the iron oxide nanoparticle and tetraethyl orthosilicate in a sol-gel process. In an embodiment, the silica coating covers substantially all of the surface of the nanoparticle. In an embodiment, the silica coating has an average thickness between about 20 nm and about 60 nm. In an embodiment, the average thickness is about 40 nm. In an embodiment, the silica coating has a substantially uniform thickness.

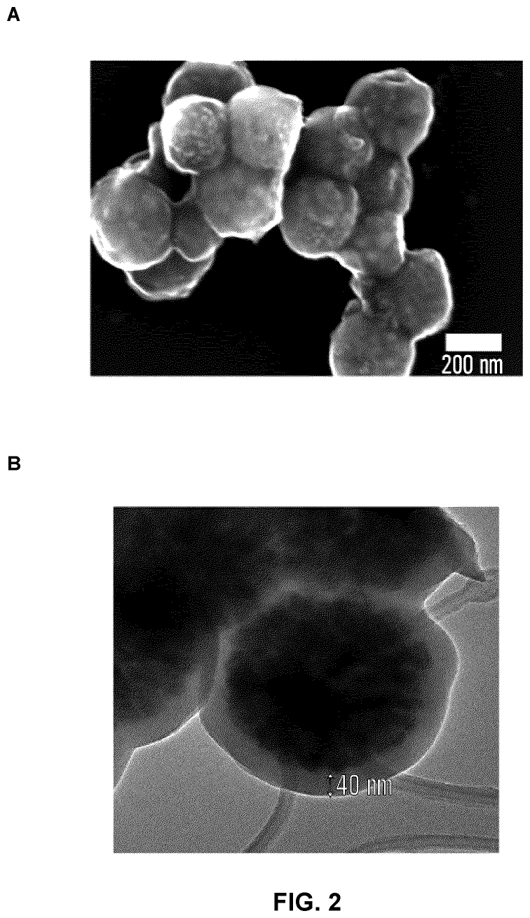

FIG. 2A shows an SEM image and FIG. 2B shows a high-resolution transmission electron microscopy (TEM) image of the core-shell structure of silica-coated iron oxide nanoparticles prepared as described above and in Example 2. An advantage of the silica shell is that it provides an inert or anti-biofouling surface with respect to DNA binding. In an embodiment, individual silica-coated nanoparticles can aggregate to form nanoparticle clusters (see e.g. FIG. 2A).

In the methods of the present disclosure, the silica-coated nanoparticle is conjugated to one or more biomolecule probes. As used herein, the term “conjugated” and its derivatives is intended to refer to the joining, linking or attachment of two or more molecules, i.e. the silica-coated nanoparticle and the biomolecule probe. The joining may be through a covalent bond or through a covalently bonded linkage. Alternatively, the joining may be a non-covalent linkage.

As used herein, the term “biomolecule probe” is intended to refer to a probe that is comprised of or consists of one or more biomolecules. In an embodiment, the biomolecule of the biomolecule probe is an oligonucleotide, a polypeptide, a bioconjugate, or an enzyme.

In a particular embodiment, the biomolecule probe is an oligonucleotide probe. The oligonucleotide probe may be designed to be partially or entirely complementary to a target nucleic acid, such a low abundant target nucleic acid as described herein. The oligonucleotide probe may be of any length suitable to capture the low abundant target oligonucleotide. In an embodiment, the oligonucleotide probe is at least 10 nucleotides in length. In an embodiment, the oligonucleotide probe has a length of 10 to 150 nucleotides. In an embodiment, the oligonucleotide probe has a length of between 50 to 150 nucleotides. In an embodiment, the oligonucleotide probe has a length of 90 to 120 nucleotides. In an embodiment, the oligonucleotide probe has a length about 10, about 15, about 20, about 25, about 30, about 35, about 40, about 45, about 50, about 55, about 60, about 65, about 70, about 75, about 80, about 85, about 90, about 95, about 100, about 110, about 115, about 120, about 125, about 130, about 135, about 140, about 145, or about 150 nucleotides. In an embodiment, the oligonucleotide probe is a primer.

In an embodiment, the biomolecule probe is a polypeptide probe. The polypeptide probe may be of any length suitable to capture a low abundant target biomolecule. In an embodiment, the peptide probe is at least 10 amino acids in length. In an embodiment, the peptide probe has a length of 10 to 1500 amino acids. In an embodiment, the peptide probe has a length of between 50 to 750 amino acids. In an embodiment, the peptide probe has a length of 100 to 300 amino acids.

In the methods of the present disclosure, the silica-coated nanoparticle is conjugated to the one or more biomolecule probes. By “conjugated”, it is meant that the biomolecule is attached to the silica-coated nanoparticle. The conjugation may be by any suitable means so long as it does not comprise a polypeptide or involve protein-protein interaction.

In an embodiment, the biomolecule is conjugated to the silica-coated nanoparticle by employing a click chemistry reaction. Click chemistry reactions are selective, efficiently performed in aqueous solvents, and produce non-harmful by-products that can be removed without the use of chromatography. Examples of click chemistry reactions include, but are not limited to, Cu(I)-catalyzed azide-alkyne cycloaddition (CuAAC), strain-promoted azide-alkyne cycloaddition (SPAAC), and strain-promoted alkyne nitrone cycloaddition (SPANC). In an embodiment, the click chemistry reaction is a Cu(I)-catalyzed azide-alkyne cycloaddition click reaction. The Cu(I)-catalyzed azide-alkyne cycloaddition click reaction is a very selective, highly specific coupling strategy for biomolecules.²⁵⁻²⁸ The click chemistry reaction can be advantageous because it does not interfere with most other organic groups present in DNA, forms triazole linkages quickly and quantitatively in a vast variety of conditions and is pH insensitive.

In an embodiment, the biomolecule is conjugated to the silica-coated nanoparticle by a triazole linkage. As used herein, the term “triazole linkage” is intended to refer to a connection between the silica-coated nanoparticle and the one or more biomolecule probes that comprises a triazole molecule. The triazole linkage between the silica-coated nanoparticle and the biomolecule probes may be formed by any means. In an embodiment, the triazole linkage is by a click chemistry reaction, such as for example and without limitation, those above. In exemplary embodiments, the click chemistry reaction is an azide-alkyne cycloaddition reaction providing a triazole linkage.

In an embodiment, the silica of the silica-coated nanoparticle may be functionalized to provide the triazole linkage upon conjugation with a functionalized biomolecule probe. As used herein, the term “functionalized” is intended to refer to a modification with a functional group to allow for conjugation between the silica-coated nanoparticle and the functionalized biomolecule probe. In an embodiment, the silica is functionalized with a first functional group and the biomolecule probe is functionalized with a second functional group capable of interacting with the first functional group. In an embodiment, the functional groups are capable of undergoing the click chemistry reaction.

In an embodiment, the functionalized silica comprises an azide as a first functional group and the functionalized biomolecule probe comprises an alkyne as a second functional group. In another embodiment, the functionalized silica comprises an alkyne as a first functional group and the functionalized biomolecule probe comprises an azide as a second functional group.

In an embodiment, the one or more biomolecule probes may be conjugated to the silica-coated nanoparticle by a spacer molecule. As used herein, the term “spacer molecule” is intended to refer to one or more molecules, such as for example an oligonucleotide sequence, which do not act as a biomolecule probe, but rather spaces the biomolecule probe further away from the silica-coated nanoparticle. In an embodiment, the spacer molecule comprises an oligonucleotide sequence that is substantially different from the oligonucleotide probe. In embodiment, the spacer molecule comprises a synthetic nucleotide sequence. In an embodiment, the spacer molecule comprises an artificial nucleotide sequence. As used herein, the term “artificial nucleotide sequence” is intended to mean a sequence that has not been identified to occur in nature.

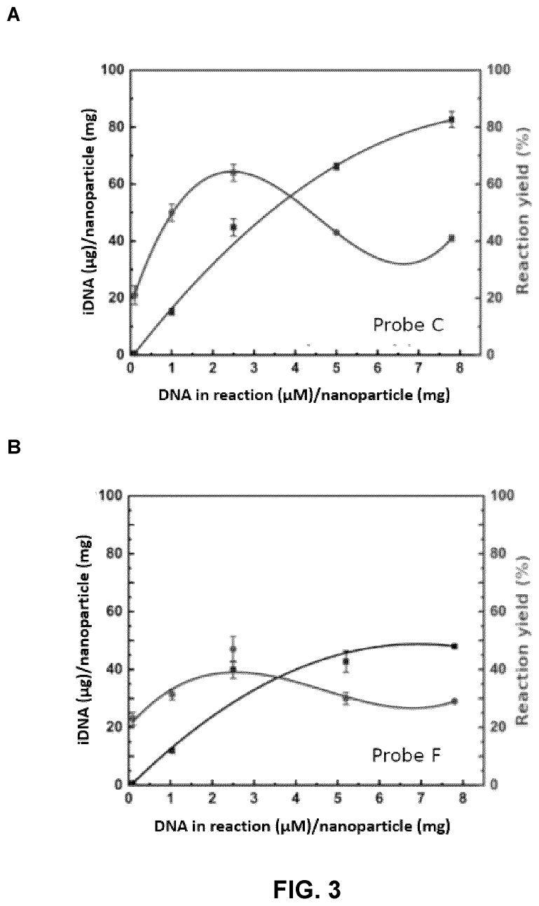

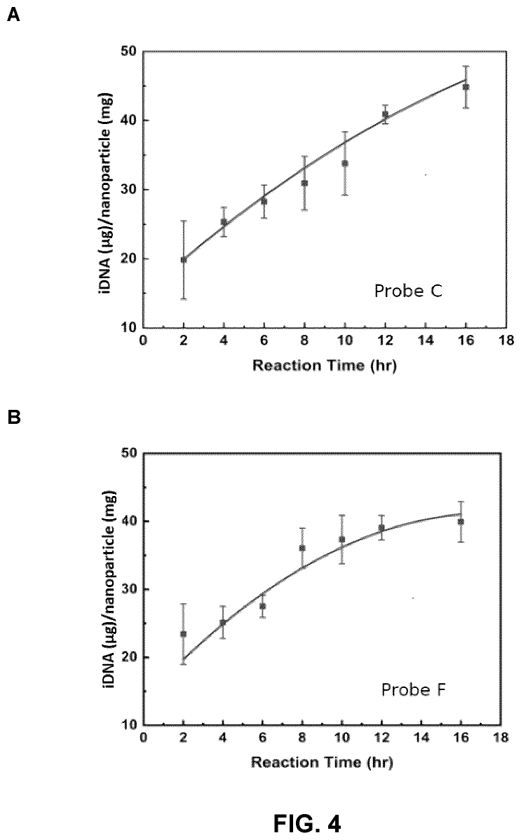

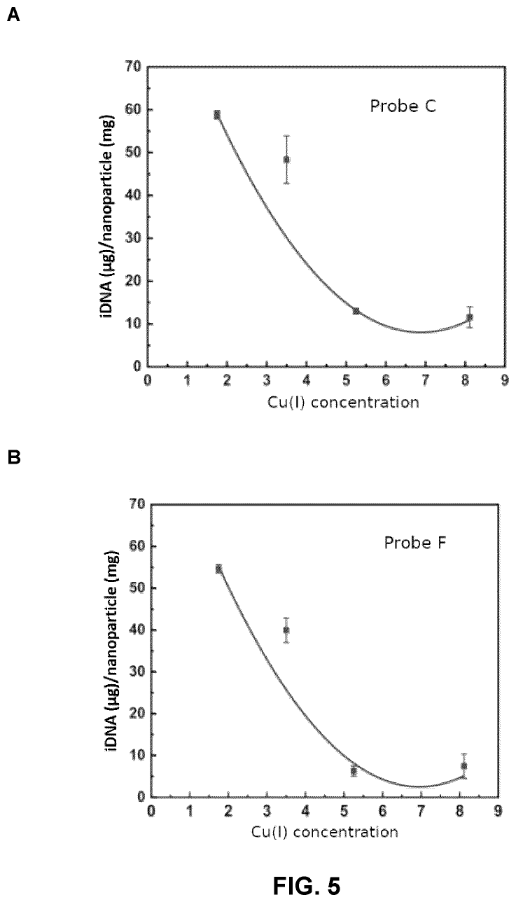

In an embodiment, the one or more biomolecule probes form a dense monolayer on the silica. As used herein, the term “dense monolayer” is meant to refer to a uniform, concentrated distribution of the one or more biomolecule probes around the silica shell. Without being bound by any particular theory, the steric hindrance and negative charge provided by the dense monolayer of the one or more biomolecule probes may reduce the likelihood of non-specific binding. In an embodiment, the extent of conjugation of the one or more biomolecule probes to the silica-coated nanoparticles is controlled by one or more of probe/nanoparticle ratio, reaction time, and Cu(I) concentration. Non-limiting examples of controlling the extent of conjugation of the one or more biomolecule probes to silica-coated iron oxide nanoparticles are show in FIG. 3, FIG. 4, FIG. 5, and Examples 2 to 4 for oligonucleotide probes.

It is contemplated herein that any given individual capture compound comprising oligonucleotide probes may have oligonucleotide probes that all comprise the same nucleotide sequence or have oligonucleotide probes with different sequences. Thus, in an embodiment, the one or more oligonucleotide probes on the capture compound comprise the same nucleotide sequence. In another embodiment, the one or more oligonucleotide probes on the capture compound comprise different nucleotide sequences.

It is also contemplated herein that any given sample of capture compounds may comprise capture compounds having different oligonucleotide probes. In an embodiment, any given capture compound within the sample will only comprise oligonucleotide probes of the same sequence, but different capture compounds within the sample will have different oligonucleotide probes. This may be particularly useful in applications involving the detection of gene panels.

In an embodiment of the methods herein in which the biomolecule probe is an oligonucleotide, the oligonucleotide probes may comprise a sequence that has at least 50%, at least 55%, at least 60%, at least 65%, at least 70%, at least 75%, at least 80%, at least 85%, at least 90%, or at least 95% complementarity to the target nucleic acid over the full length of the target low abundant biomolecule (e.g. nucleic acid). In an embodiment, the oligonucleotide probe comprises a sequence that is 100% complementary to a low abundant target nucleic acid over the full length of the low abundant target nucleic acid. As used herein, complementarity has its ordinary meaning in the art. Each nucleotide has a nitrogenous base, and each nitrogenous base can pair with the nitrogenous base from another different nucleotide. Generally speaking, with respect to DNA, the bases that are complementary are: A with T and C with G. Complementarity can be determined by comparing each position in the aligned sequences and determining the number or percentage of complementary nucleotides in reference to the entire length of the aligned sequence. Optimal alignment of sequences may be conducted using a variety of algorithms, as are known in the art, such as for example the BLAST algorithm.

The calculation of complementarity may be in reference to a naturally occurring sequence of a target nucleic acid. However, it is contemplated that the target nucleic may comprise one or more unknown mutations or modifications, such that the actual complementarity may not be as designed or expected.

In an embodiment, the biomolecule probe is an oligonucleotide probe to a target nucleic acid as described herein. In an embodiment, the oligonucleotide probe is a probe to a nucleic acid of an infectious agent. In an embodiment, the oligonucleotide probe is a probe to a nucleic acid of a hepatitis C virus genome. In an embodiment, the oligonucleotide probe is a probe to a nucleic acid of a tuberculosis genome. In an embodiment, the oligonucleotide probe is a probe to a nucleic acid of a coronavirus genome.

In an embodiment, the biomolecule probe of the present disclosure may comprise or consist of one or more of the following nucleotide sequences (SEQ ID NO: 1-8). In an embodiment, the biomolecule probe is an oligonucleotide probe comprising one of SEQ ID NO: 1-8. In an embodiment, the oligonucleotide probe consists of one of SEQ ID NO: 1-8:

(SEQ ID NO: 1)
TTTGGGCGTGCCCCCGCAAGACTGCTAGCCGAGTAGTGTTGGGTCGCGAA
AGGCCTTGTGGTACTGCCTGATAGGGTGCTTGCGAGTGCC;
(SEQ ID NO: 2)
CCGGGAGGTCTCGTAGACCGTGCACCATGAGCACGAATCCTAAACCTCAA
AGAAAAACCAAACGTAACACCAACCGTCGCCCACAGGACG;
(SEQ ID NO: 3)
GCAATTGGTTCGGTTGTACCTGGATGAACTCAACTGGATTCACCAAAGTG
TGCGGAGCGCCCCCTTGTGTCATCGGAGGGGTGGGCAACA;
(SEQ ID NO: 4)
TGTCCACCACACAGTGGCAGGTCCTTCCGTGTTCTTTCACGACCCTGCCA
GCCTTGTCCACCGGCCTCATCCACCTCCACCAGAACATTG;
(SEQ ID NO: 5)
ACCATGTTTCCCCCACGCACTACGTGCCGGAGAGCGATGCAGCCGCCCGC
GTCACTGCCATACTCAGCAGCCTCACTGTAACCCAGCTCC;
(SEQ ID NO: 6)
GCTCCGGTTCCTGGCTAAGGGACATCTGGGACTGGATATGCGAGGTGCTG
AGCGACTTTAAGACCTGGCTGAAAGCCAAGCTCATGCCAC;
(SEQ ID NO: 7)
TTATAACATCATGCTCCTCCAACGTGTCAGTCGCCCACGACGGCGCTGGA
AAGAGGGTCTACTACCTTACCCGTGACCCTACAACCCCCC;
or
(SEQ ID NO: 8)
CCTGGCTAGGCAACATAATCATGTTTGCCCCCACACTGTGGGCGAGGATG
ATACTGATGACCCATTTCTTTAGCGTCCTCATAGCCAGGG.

The capture compounds as disclosed herein may be used in any number of applications for capturing, separating, and/or enriching a low abundant target biomolecule (e.g. a low abundant target nucleic acid) in or from a sample.

In an embodiment, the capture compound may be capable of providing improved capture specificity of one or more target nucleic acids of a gene panel. In an embodiment, the capture compounds of the present disclosure may be used for improving capture specificity of one or more target nucleic acids of a gene panel. In such applications, different capture compounds can be used that comprise oligonucleotide probes to different target nucleic acids within the gene panel.

In an embodiment, the capture compounds of the present disclosure may be used for capturing a low abundant target nucleic acid in the methods disclosed herein. In an embodiment, the capture compounds of the present disclosure may be used for capturing a target other than a nucleic acid, for example a protein. In an embodiment, the capture compounds of the present disclosure may be used for capturing a target nucleic acid of a human genome (whether mutated or not) and/or an expression product thereof (e.g. mRNA).

In an embodiment, the capture compounds of the present disclosure may be used for enrichment of a low abundant target biomolecule in a biological sample by downstream detection, quantification, or amplification assays. Non-limiting examples of the downstream detection, quantification or amplification assays include real-time polymerase chain reaction (PCR), loop-mediated isothermal amplification (LAMP), quantitative PCR (qPCR), or a sequencing method. In an embodiment, the low abundant target is a low abundant target nucleic acid of a viral genome, a bacterial genome, or a fungal genome; comprises a mutated or non-mutated nucleotide sequence of a nucleic acid of a human genome; or is a synthetic oligonucleotide.

In an embodiment, the method for capturing a low abundant target biomolecule comprises providing two or more capture compounds, each capture compound comprising a different oligonucleotide probe and the two or more capture compounds capable of detecting and/or capturing different target nucleic acids of a gene panel. In an embodiment, providing the two or more capture compounds is for improving capture specificity of one or more target nucleic acids of the gene panel. In an embodiment, the sample is a biological sample comprising a low abundant target nucleic acid as the target nucleic acid.

In an embodiment, the method is for capturing a low abundant target nucleic acid of a viral genome, a bacterial genome, or a fungal genome; or which comprises a mutated or non-mutated nucleotide sequence of a nucleic acid of a human genome. In an embodiment, the method is for capturing a low abundant target nucleic acid of a hepatitis C virus genome. In an embodiment, the method is for capturing a low abundant target nucleic acid or a target protein of a coronavirus genome (e.g. a SARS CoV-2 virus genome).

In an embodiment, the method is for capturing a low abundant viral, bacterial, or fungal protein. In an embodiment, the method is for capturing a low abundant target protein of a hepatitis C virus. In an embodiment, the method is for capturing a low abundant target protein of a coronavirus (e.g. a SARS CoV-2 virus).

In some embodiments of the present disclosure, some or all of the incubating in the methods disclosed herein is done under high stringency conditions. As used herein, the term “high stringency conditions” is intended to refer to conditions under which the oligonucleotide probe is more likely to bind to targets of high complementarity, thereby improving specificity. An advantageous aspect of the capture compounds of the present disclosure is that their improvement in avoiding non-specific binding may negate the need for high stringency conditions all together and/or will improve the efficiency of capture under these conditions, which is particularly useful for low abundant targets. High stringency conditions can be achieved by controlling parameters such as temperature, pH, and salt concentrations.

In an embodiment, the high stringency conditions comprise hybridization in a buffer comprising 10× SSPE, 10× Denhardt's solution, 10 mM EDTA and 0.2% SDS at between about 65° C. and about 70° C. The person skilled in the art will appreciate that stringency conditions can be optimized for a given system and that other buffer solutions may provide the desired high stringency conditions. In an embodiment, the high stringency conditions comprise washing in a wash buffer comprising 0.1× SSC, 1% SDS at about 65° C. In an embodiment, the high stringency conditions comprise washing in a wash buffer comprising 1× SCC, 0.1% SDS at about 65° C. In an embodiment, the high stringency conditions comprise washing in each of a wash buffer comprising 0.1× SSC, 1% SDS at about 65° C. and a wash buffer comprising 1× SCC, 0.1% SDS at about 65° C. The skilled person will appreciate that other wash buffers may be used to achieve suitable high stringency conditions.

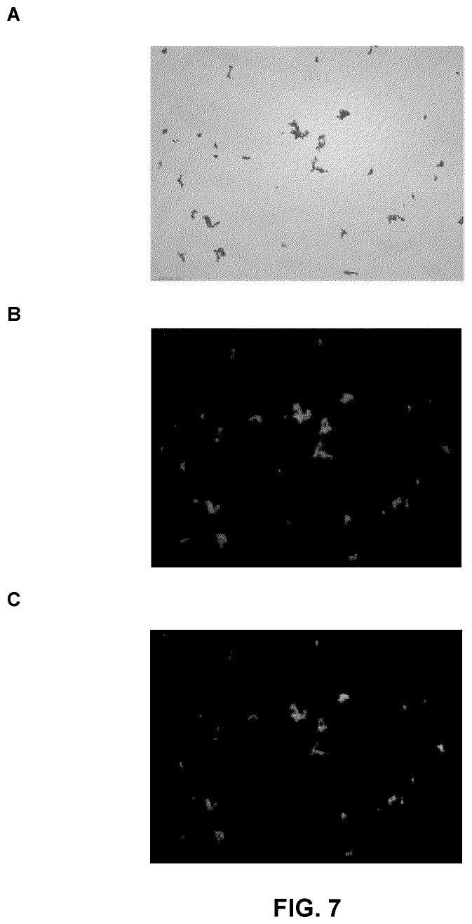

FIG. 7 shows a non-limiting example of a hybridized DNA probe modified silica-coated iron oxide nanoparticle under fluorescent microscope. FIG. 7A is a bright field image, FIG. 7B is an image under an Alex Fluor 488 (green) filter, and FIG. 7C is an image under a Cy5 (red) filter.

In some embodiments of the present disclosure, the method for capturing a low abundant target biomolecule further comprises adding one or more blocking agents to the sample prior to incubating the sample with the capture compound. Non-limiting examples of blocking agents include human cot-1 DNA, salmon sperm DNA, and blocking oligonucleotides complementary to library adaptor sequences.

In an embodiment, the method for capturing a low abundant target biomolecule further comprises isolating or separating the low abundant target biomolecule from the sample. In an embodiment, the isolating or separating is by magnetic attraction between a magnetic source and the capture compound. In such embodiments, the nanoparticle is preferably a magnetic nanoparticle. In another embodiment, the isolating or separating is by centrifugation or filtration, or both.

In an embodiment, the method for capturing a low abundant target biomolecule further comprises dissociating the low abundant target biomolecule from the capture compound. The dissociating may be by any suitable means to unbind the target biomolecule from the capture compound, including by chemical reaction and/or by heating. Dissociating the low abundant target biomolecule advantageously allows for subsequent use of the target biomolecule, including for example for performing diagnostic and/or prognostic experiments. In an embodiment, dissociating the low abundant target biomolecule allows for further purification of the target biomolecule which may be useful or even necessary for certain downstream processes (e.g. qPCR, LAMP, etc.), due to the potential inhibition by various factors when the target biomolecule is attached to the nanoparticle.

In an embodiment, the method for capturing a low abundant target biomolecule has improved efficiency in capture of the low abundant target biomolecule as compared to a capture assay that does not employ the capture compound. By “improved efficiency”, it is meant for example that the capture compounds and methods of the present disclosure provide improved specificity for the low abundant target biomolecule, with a reduction in non-specific binding. In an embodiment, the efficiency of capture as measured by the copy number of the target biomolecule that is captured may be improved by at least 1.5-fold, 2-fold, 5-fold, 10-fold, 25-fold, 50-fold, 75-fold, 100-fold, 125-fold, 150-fold, 200-fold or more.

In an embodiment, the method for capturing a low abundant target biomolecule has improved efficiency as compared to a capture assay that does not employ the capture compound. In an embodiment, the improved efficiency comprises improved specificity for the target nucleic acid. In an embodiment, the improved efficiency comprises an improved ability to capture a low abundant target biomolecule and/or permit enrichment of a low abundant target biomolecule (e.g. by PCR). In an embodiment, efficiency may be measured by enrichment (e.g. copy number) of the low abundant target biomolecule (e.g. nucleic acid). In an embodiment, the methods of the present disclosure are at least 10-times, at least 25-times, at least 50-times, at least 75-times, at least 100-times, at least 150-times, at least 200-times more effective as compared to a capture assay that does not employ the capture compound (e.g. Streptavidin).

In some embodiments, the present disclosure provides a method of enriching a low abundant target biomolecule in a biological sample, comprising: providing an unbound capture compound compound comprising a silica-coated nanoparticle conjugated to one or more biomolecule probes; incubating the capture compound with a biological sample comprising the low abundant target biomolecule; and performing an amplification to enrich the low abundant target nucleic biomolecule in the biological sample. In an embodiment, the amplification reaction is a polymerase chain reaction (PCR) or a loop-mediated isothermal amplification (LAMP). In a particular embodiment, the amplification reaction is PCR. In an embodiment, the low abundant target biomolecule is a nucleic acid as descried elsewhere herein.

The capture compound may be any of the capture compounds disclosed elsewhere herein. In an embodiment, the silica-coated nanoparticle is a silica-coated iron oxide nanoparticle. In an embodiment, the one or more biomolecule probes form a dense monolayer on the silica. In an embodiment, the one or more biomolecule probes are oligonucleotide probes. The one or more oligonucleotide probes may comprise the same nucleotide sequence or have oligonucleotide probes with different sequences. In an embodiment, the one or more oligonucleotide probes have a length of 90 to 120 nucleotides. In an embodiment, the one or more oligonucleotide probes comprise a nucleotide sequence that has at least 70% complementarity to a low abundant target nucleic acid over the full length of the low abundant target nucleic acid. In an embodiment, the one or more oligonucleotide probes comprise a nucleotide sequence that is 100% complementary to a low abundant target nucleic acid over the full length of the low abundant target nucleic acid.

In an embodiment, the PCR is an on-beads PCR. In an embodiment, the method of enriching a low abundant target biomolecule in a biological sample further comprises performing a quantitative polymerase chain reaction (qPCR) on an amplification product of the on-beads PCR. In an embodiment, to perform the enrichment, the target biomolecule is dissociated from the nanoparticle before performing the amplification, such as prior to performing amplification by qPCR or a loop-mediated isothermal amplification (LAMP).

In an embodiment, in respect of the oligonucleotide probes of SEQ ID NOs: 1-8, the qPCR primer set may comprise:

(SEQ ID NO: 9)
CGGAATTGCCAGGACGAC
(SEQ ID NO: 10)
GGATTCGTGCTCATGGTGC
(SEQ ID NO: 11)
CTGCTAGCCGAGTAGTGTTGG
(SEQ ID NO: 12)
GGAACTTGACGTCCTGTGG
(SEQ ID NO: 13)
GGTATATTGCTTCACTCCCAGC
(SEQ ID NO: 14)
CATCCAGGTACAACCGAACC
(SEQ ID NO: 15)
GTCAGGATGTACGTGGGAGG
(SEQ ID NO: 16)
CGTGAAAGAACACGGAAGG
(SEQ ID NO: 17)
CAGCCTCACTGTAACCCAGC
(SEQ ID NO: 18)
ACACAAAGGGAATCCCAGG
(SEQ ID NO: 19)
TAAGGGACATCTGGGACTGG
(SEQ ID NO: 20)
GTCCAGTGATCTCAGCTCCAC
(SEQ ID NO: 21)
TCCTCCAACGTGTCAGTCG
(SEQ ID NO: 22)
GGTCATCAGTATCATCCTCGC
(SEQ ID NO: 23)
GGAGACAGCAAGACACACTCC
(SEQ ID NO: 24)
CTATGGAGTAGCAGGCTCCG

In embodiment, the method of enriching a low abundant target biomolecule in a biological sample further comprises isolating or separating the low abundant target biomolecule from the biological sample by magnetic attraction between a magnetic source and the capture compound. In an embodiment, the isolating or separating is by centrifugation, filtration, or both.

In an embodiment, the method of enriching a low abundant target biomolecule in a biological sample is for enriching a low abundant target nucleic acid of a viral genome, a bacterial genome, or a fungal genome; or a low abundant target nucleic acid which comprises a mutated or non-mutated nucleotide sequence of a nucleic acid of a human genome. In an embodiment, the method is for enriching a low abundant target nucleic acid of a hepatitis C virus genome. In an embodiment, the method is for enriching a low abundant target nucleic acid of a coronavirus genome (e.g. SARS CoV-2).

In an embodiment, the method of enriching a low abundant target biomolecule in a biological sample is for enriching a low abundant target protein of a virus, a bacteria, or a fungus. In an embodiment, the method is for enriching a low abundant target protein of a hepatitis C virus. In an embodiment, the method is for enriching a low abundant target protein of a coronavirus (e.g. SARS CoV-2).

In some embodiments of the present disclosure, some or all of the incubating in the method of enriching a low abundant target biomolecule in a biological sample is under high stringency conditions. In an embodiment, the high stringency conditions comprise hybridization in a buffer comprising 10× SSPE, 10× Denhardt's solution, 10 mM EDTA and 0.2% SDS at between about 65° C. and about 70° C. In an embodiment, the the high stringency conditions comprise washing in a wash buffer comprising 0.1× SSC, 1% SDS at about 65° C. In an embodiment, high stringency conditions comprise washing in a wash buffer comprising 1× SCC, 0.1% SDS at about 65° C. In an embodiment, high stringency conditions comprise washing in each of a wash buffer comprising 0.1× SSC, 1% SDS at about 65° C. and a wash buffer comprising 1× SCC, 0.1% SDS at about 65° C. The skilled person will appreciate that other wash buffers may be used to achieve the high stringency conditions.

In embodiments of the present disclosure, the method of enriching a low abundant target biomolecule in a biological sample further comprises adding one or more blocking agents to the biological sample prior to incubating the sample with the capture compound. In some embodiments, the method of enriching a low abundant target biomolecule in a biological sample is a method having improved efficiency in enriching the low abundant target biomolecule as compared to an assay that does not employ the capture compound. In an embodiment, the improved efficiency comprises improved specificity for the target nucleic acid.

In particular embodiments, the capture and enrichment methods disclosed herein are particularly advantageous in that the improvement in specificity for low abundant target nucleic acids significantly reduces the cost of downstream sequencing. By having improved target specificity, there is a significant reduction in non-target capture which, in turn, significantly reduces undesirable sequencing of extraneous or non-target DNA. This is a major advantage of the methods disclosed herein.

In some embodiments, the present disclosure provides a method for diagnosis of a disease and/or disorder associated with an infectious agent or a mutated or non-mutated nucleotide sequence of a nucleic acid of a human genome. As used herein, “diagnosis” is intended to refer not only determining whether or not an individual has a disease or disorder, but also to determining the likelihood of an individual developing the disease or disorder. Further, in an embodiment, by determining the likelihood of an individual developing a disease or disorder, the diagnostic methods may be used to pre-emptively reduce the likelihood of disease in the future.

In an embodiment, the method for diagnosis of a disease and/or disorder associated with an infectious agent or a mutated or non-mutated nucleotide sequence of a nucleic acid of a human genome, comprises: providing a biological sample from a subject; and capturing a low abundant target biomolecule of an infectious agent and/or a low abundant target nucleic acid which comprises a mutated or non-mutated nucleotide sequence of a nucleic acid of a human genome by using a capture compound comprising comprising a silica-coated nanoparticle conjugated to one or more biomolecule probes in a capture assay.

As will be appreciated, the step of capturing the low abundant target biomolecule of an infectious agent and/or the low abundant target nucleic acid which comprises a mutated or non-mutated nucleotide sequence of a nucleic acid of a human genome, may be performed in a similar manner as described herein for other capture methods. Likewise, the same or different capture compounds, reaction conditions, etc. may be used in the methods for diagnosis of a disease and/or disorder.

In particular embodiments, the capture methods disclosed herein are advantageous in diagnosis of a disease and/or disorder because the ability to capture low abundant target biomolecules may provide for early detection and/or more accurate diagnosis of state of the disease and/or disorder.

In this regard, in other embodiments, the present disclosure provides a method for prognosis of a disease and/or disorder associated with an infectious agent or a mutated or non-mutated nucleotide sequence of a nucleic acid of a human genome, the method comprising:
providing a biological sample from a subject;
capturing a low abundant target biomolecule of an infectious agent and/or a low abundant target nucleic acid which comprises a mutated or non-mutated nucleotide sequence of a nucleic acid of a human genome using a capture compound comprising a silica-coated nanoparticle conjugated to one or more biomolecule probes in a capture assay; and
detecting for a quantity of the low abundant target biomolecule and/or the low abundant target nucleic acid, wherein either:
   an elevated or reduced level of the low abundant target biomolecule and/or the low abundant target nucleic acid in the biological sample as compared to a predefined value is indicative of a poor prognosis or active disease state; or
   an elevated or reduced level of the low abundant target biomolecule and/or the low abundant target nucleic acid in the biological sample as compared to an earlier sample from the subject is indicative of a poor prognosis or active disease state.

Here again, as will be appreciated, the step of capturing the low abundant target biomolecule of an infectious agent and/or the low abundant target nucleic acid which comprises a mutated or non-mutated nucleotide sequence of a nucleic acid of a human genome, may be performed in a similar manner as described herein for other capture methods. Likewise, the same or different capture compounds, reaction conditions, etc. may be used in the methods for prognosis of a disease and/or disorder.

In particular embodiments, the capture methods disclosed herein are advantageous in prognosis of a disease and/or disorder because the ability to capture low abundant target biomolecules may provide for better characterization of state of disease and change in disease status. For example, the ability to capture low abundant target biomolecules by less complex methods may permit earlier detection and better monitoring of disease condition, and also provide a more accurate prognosis of a subject having been cured since the ability to capture low abundant targets will permit more reliable findings and conclusions on irradiation of the infectious agent.

In conjunction with the methods disclosed herein, the present disclosure also provides uses of the capture compounds for the capture and/or enrichment of low abundant target biomolecules. This includes, for example and without limitation, use of a capture compound comprising a silica-coated nanoparticle conjugated to one or more biomolecule probes for:
improving capture specificity of one or more low abundant target nucleic acids of a gene panel;
capturing a low abundant target biomolecule in a biological sample; and/or
enrichment of a low abundant target biomolecule in a biological sample by a downstream amplification assay.

In some embodiments, the present disclosure provides a kit for preparing the capture compound of claim 1, the kit comprising: a silica-coated nanoparticle; 3-azidopropyltriethoxysilane for azide functionalization of the silica; and instructions for conjugating a 5′-Hexynyl-modified oligonucleotide probe to the silica-coated nanoparticle. In some embodiments, the silica-coated nanoparticle is a silica-coated iron oxide nanoparticle. In some embodiments, the kit further comprises the 5′-Hexynyl-modified oligonucleotide probe.

In some embodiments, the present disclosure provides a capture assay kit for detection of a low abundant target biomolecule, the capture assay kit comprising: a capture compound comprising a silica-coated nanoparticle conjugated to one or more biomolecule probes and one or more reagents for low abundant capture. The reagents may, for example, include hybridization and wash buffers in accordance with the present disclosure.

### EXAMPLES

### Example 1—Preparation of a Silica-Coated Iron Oxide Capture Compound

Synthesis of Iron Oxide Nanoparticles

Superparamagnetic iron oxide nanoparticles were synthesized as per Tian and collaborators²⁹, with modifications.

An iron precursor solution was prepared by dissolving FeCl₃·6H₂O (0.8 g; Sigma-Aldrich) in ethylene glycol (30 mL; Sigma-Aldrich). Sodium acetate (1.189 g; Sigma-Aldrich) was added to adjust the pH and the mixture was stirred for 20 min. The mixture was transferred to an autoclave chamber and heated at 200° C. for 8 h. The autoclave was naturally cooled and iron oxide nanoparticles were collected by magnetic separation. Nanoparticles were washed 3× with water and ethanol, and freeze-dried.

The size and morphology of the iron oxide nanoparticles were determined by field emission high-resolution scanning electron microscopy (SEM) on a Hitachi-54800 HR at 30 KV (FIG. 1A) and transmission electron microscopy JEOL TEM-2200FS imaging. Samples were prepared by dropping the nanoparticle suspension on a 400-mesh carbon grid and dried in a vacuum oven for 2 h. The average diameter of the iron oxide nanoparticles was found to be about 235 nm (FIG. 1B).

Coating of Iron Oxide Nanoparticles

Iron oxide nanoparticles were coated with an approximately 40 nm silica shell via a hydrolysis-condensation reaction of tetraethyl orthosilicate (TEOS) in a sol-gel process.³⁰ A suspension of iron oxide nanoparticles (1 mg/mL) in a solution of water, ethanol, and ammonium hydroxide was prepared. The mixture was sonicated to disperse the magnetic particles and TEOS (0.05 mg; Sigma-Aldrich) was added dropwise under sonication to initiate the coating process. The reaction was vigorously stirred for 6 h. The resulting silica-coated iron oxide nanoparticles were collected by magnetic separation and freeze-dried.

The core-shell structure of the silica-coated iron oxide nanoparticles was determined to have an average 40 nm silica coating as characterized by field emission high-resolution scanning electron microscopy (SEM) on a Hitachi-54800 HR at 30 KV, and transmission electron microscopy JEOL TEM-2200FS imaging (FIG. 2). Samples were prepared by dropping the nanoparticle suspension on a 400-mesh carbon grid and dried in a vacuum oven for 2 h.

X-ray powder diffraction (XRD) patterns were collected using a Rigaku XRD Ultima IV. The XRD pattern showed five characteristic peaks and intensities indicated that the iron oxide nanoparticles were pure Fe₃O₄ with spinal structure.

Magnetization measurements were performed using a Quantum Design 9T-PPMS dc magnetometer/ac susceptometer over the range of −30 to 30 KOe at 300 K and a high saturation magnetization of 82 emu/g⁻¹ was observed.

Azide Functionalization of Silica-Coated Iron Oxide Nanoparticles

Azide functionalization groups were added to the silica-coated surface through a 3-azidopropyl triethoxysilane deposition.³¹

Briefly, to a suspension of dried silica-coated iron oxide nanoparticles (5 mg) in tetrahydrofuran (THF; Sigma-Aldrich) under N₂ protection, was added 3-azidopropyl triethoxysilane (4 mg) under continuous stirring. The mixture was stirred for 2 days at room temperature. The resulting functionalized silica-coated iron oxide nanoparticles were collected by magnetic separation, washed 3× with THF and ethanol three times, and freeze-dried.

For infrared characterization of the functionalized layer, Fourier-transform infrared (FT-IR) spectroscopy characterization was carried out using FT-IR Agilent FTS 7000 in the range of 400-4000 cm⁻¹. Adsorption bands at approximately 582 cm⁻¹ were assigned to the Fe—O group in the Fe₃O₄ nanoparticles. Formation of the silica coating was confirmed by the Si—O—Si absorption at 1082 cm⁻¹ attributed to Si—O—Si in Fe₃O₄©SiO2 and Fe₃O₄©SiO₂©N₃. The azide functional group on the nanoparticle surface was confirmed by the N₃ stretching peak at 2096 cm⁻¹. The thick silica shell prevented observation of a strong peak corresponding to the azide group. Therefore, the azide functionalized silica-coated iron oxide nanoparticles were further analyzed by X-ray photoelectron spectroscopy (XPS).

XPS spectra were acquired on a Kratos AXIS 165 electron spectrometer with 150 W monochromatized Al Ka radiation (1486.6 eV), where all peaks were referred to the signature C1s peak for adventitious carbon at 284.8 eV. A peak at 398 eV was observed, which corresponds to N1s peak and further confirmed the N3 was installed on the surface. In addition, peaks at 710 eV, 530 eV, 282 eV, 102 eV were observed, representing iron, oxygen, carbon, and silicon, respectively (data not shown).

Click Reaction of Azide Functionalized Silica-Coated Iron Oxide Nanoparticles with DNA Probes

DNA probe-clicked silica-coated iron oxide nanoparticles were prepared via a Cu(I)-catalyzed azide-alkyne cyclo-addition (CuAAC) reaction utilizing azide-functionalized silica-coated iron oxide nanoparticles (azide-NP) and alkyne modified oligonucleotides. In a 500 μL test tube, 24 μM 20bp 3′-fluorescein-labeled 5′-hexynyl modified DNA probe (IDT DNA; 10 μL) in water was mixed with 2 M triethylammonium acetate buffer (3 μL; pH=7.0) and dimethyl sulfoxide (DMSO; 10 μL). To this solution, azide-NP in a 1:1 solution of DMSO and water (10 μL; 9.6 mg/mL) and 5 mM ascorbic acid in water (3 μL) were added, followed by bubbling with argon for 30 s. A 10 mM Cu(II)-tris((1-benzyl-4-triazolyl)methyhamine (TBTA) solution in 55% DMSO (Lumiprobe; 3.5 μL) was added under argon. The tube was sealed and the reaction was left on a rotator for overnight at room temperature. The supernatant was removed and the DNA probe-clicked silica-coated iron oxide nanoparticles were re-dispersed in water. Removal of supernatant and re-dispersion was repeated five times to remove any residual reagents. The DNA probe-clicked silica-coated iron oxide nanoparticles were stored in water at 4° C. Storage under these conditions resulted in stability for 1 y.

To determine the density of DNA probes on the nanoparticle surface, the supernatant and three washes were collected right after the reaction. The DNA concentration was estimated using Quant-iTTM OliGreen ssDNA assay kit (Thermo Fisher Scientific).

### Example 2—Controlling Probe Immobilization by [Probe]/[Nanoparticle] Ratio

The density of probes on the beads surface has an influence on capture efficiency. To control this density, the amount of DNA in the reaction, the reaction times, and the Cu(I) concentrations were optimized. These experiments were done using two probes that bind to different regions of the HCV genome, named probe C and probe F (Table 2).

Various concentrations of the DNA probes were used to investigate the effect on conjugation efficiency, including 0.1, 1, 2.5, 5.2, 7.8 μM (Table 1). It was found that probe C has a saturation point for iDNA/nanoparticle of 82.69 μg/mg (FIG. 3A), while probe F has a saturation point for iDNA/nanoparticle of 47.98 DNA μg/mg (FIG. 3B). As shown in the right Y-axis of the plots in FIG. 3, the yield of DNA conjugation has a peak point of 64(±3)% for probe C (FIG. 3A) and 47.54(±3.03)% for probe F (FIG. 3B). The yields peaked at 2.4 μM DNA concentration for both high and low binding probes, so this concentration was used for subsequent experiments.

TABLE 1
Saturations and Reaction Yields Varying Probe Concentrations.

| DNA in reaction (μM)/nanoparticle (mg) ratio | | 0.1 | 1 | 2.5 | 5.2 | 7.8 |
|---|---|---|---|---|---|---|
| Probe C | iDNA/nanoparticle (μg/mg) | [symbol] | [symbol] | [symbol] | [symbol] | [symbol] |
| | Yield ( [symbol] ) | [symbol] | [symbol] | [symbol] | [symbol] | [symbol] |
| Probe F | iDNA/nanoparticle (μg/mg) | [symbol] | [symbol] | [symbol] | [symbol] | [symbol] |
| | Yield ( [symbol] ) | [symbol] | [symbol] | [symbol] | [symbol] | [symbol] |

[symbol]

indicates data missing or illegible when filed

### Example 3—Controlling Probe Immobilization by Reaction Time

Using the click reaction conditions of Example 1 and 2, various reaction times were used to study the impact on DNA immobilization using a DNA (μM)/nanoparticle (mg) ratio of 2.4. As shown in FIG. 4, by increasing the reaction time DNA immobilization increased. For probe F, the amount of iDNA did not increase significantly after 10 h of incubation (FIG. 4B), but for probe C the amount of iDNA increased over time almost in a linear fashion (FIG. 4A).

### Example 4—Controlling Probe Immobilization by Cu(1) Concentration

Using the click reaction conditions of Example 1 and 2, various Cu concentrations were used to study the impact on DNA immobilization using a DNA (μM)/nanoparticle (mg) ratio of 2.4. As shown in FIG. 5, the lowest Cu concentration resulted in highest conjugation reaction efficiency. Results for probe C are shown in FIG. 5A and for probe F are shown in FIG. 5B.

### Example 5—Hybridization of a Silica-Coated Iron Oxide Capture Compound

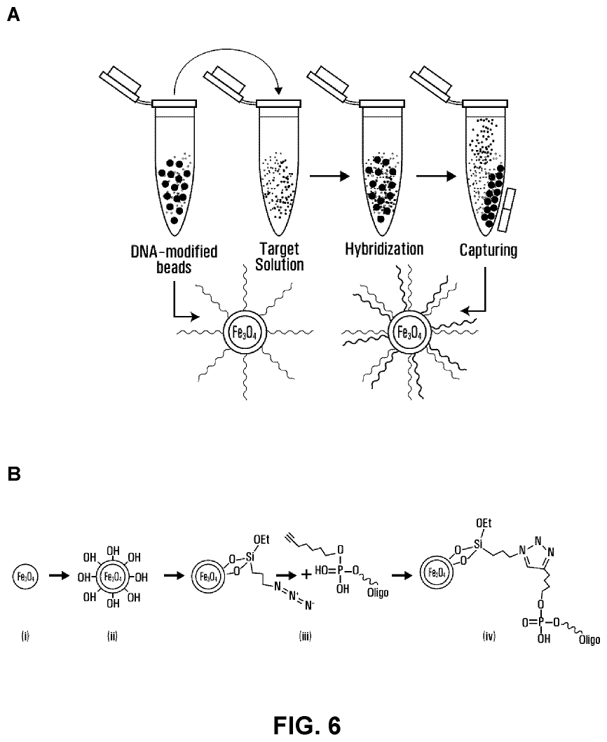

As disclosed herein, silica-coated nanoparticles (coupled to DNA probes) provide an inert surface that reduces off-target hybridization (FIG. 6A). As described in Example 1, iron oxide silica-coated nanoparticles were prepared using a solvothermal reaction, followed by a silica coating, azide functionalization, and conjugation of DNA probes using click chemistry (FIG. 6B). Click chemistry was achieved by adding a 5′-Hexynyl modification that introduces a 5′-terminal alkyne group into DNA, which readily reacts with azide in the presence of copper, forming stable 1,2,3-triazole bonds. The iron oxide cores herein have an average diameter of 235±20 nm (FIG. 1A and 1B), with an additional 40 nm shell formed by the silica coating (FIG. 2A and 2B). Individual particles can cluster.

The silica-coated iron oxide capture compound of Example 1, comprising a 20bp 3′-fluorescein-labeled 5′-Hexynyl oligonucleotide probe, was hybridized with the complementary 5′-Cy5 labeled (red-20bp). Hybridization buffer (containing 10× SSPE, 10× Denhardt's solution, 10 mM EDTA, 0.2% SDS and 20U RNase block) was freshly prepared and mixed with the probe-clicked silica-coated iron oxide nanoparticles, then pre-warmed to 65° C. and transferred to the complementary 5′-Cy5 labeled (red-20bp) mix to make a hybridization mix. The final hybridization mix was incubated at 65° C. for 24 h and slowly cooled to room temperature. Supernatant was removed and DNA probe-clicked silica-coated iron oxide nanoparticles were washed 3 times with wash buffer (0.1× SSC, 0.1% SDS) at 65° C. for 10 min each. Wash buffer was removed and the DNA probe-clicked silica-coated iron oxide nanoparticles were re-suspended with ultra-pure water. The resulting hybridized capture compound was analyzed under a fluorescent microscope under (a) bright field; (b) an Alex Fluor 488 (green) filter, labeled DNA probe IONPs; (c) Cy5 (red), labeled complementary DNA IONPs (FIG. 7). Confirmation of probe-target hybridization was observed as shown in FIG. 7B and 7C.

### Example 6—Preparation of Silica-Coated Iron Oxide Capture Compounds for Hepatitis C Virus (HCV) Targets

Silica-coated iron oxide capture compounds were prepared as described in Example 1 using 90-bp long probe sequences (Table 2) designed based on the reference genomic sequence (gi#: 22129792) of HCV genotype 1 (excluding U3 region because of repeats). The main design criteria included: (1) melting temperature within the range of hybridization temperature (65° C.) plus 15 to 25° C.; (2) GC content within 40 to 65%; (3) no significant similarity to human genomic sequence using blastn; and (4) no stable secondary structure (ΔG value higher than −9.0 kcal/mol) in hybridization conditions. Eight probes for different HCV regions (A to H; Table 2) were selected to evaluate the silica-coated iron oxide nanoparticles of Example 1 against commercial capture assays (Dynabeads MyOne streptavidin T1; Thermo Fisher Scientific). Two sets of the 8 probes (A to H; Table 2) were synthesized. One set was modified with 5′ hexynyl for the silica-coated iron oxide nanoparticles (capture compounds). The other set was modified with 5′ biotin for use with Dynabeads.

TABLE 2
Capture Probe Sequences

| | | |
|---|---|---|
| Capture | TTTGGGCGTGCCCCCGCAAGACTGCTAGCCGAGTAGTGTTGGGTCGCG | SEQ ID NO: 1 |
| probe A | AAAGGCCTTGTGGTACTGCCTGATAGGGTGCTTGCGAGTGCC | |
| Capture | CCGGGAGGTCTCGTAGACCGTGCACCATGAGCACGAATCCTAAACCTC | SEQ ID NO: 2 |
| probe B | AAAGAAAAACCAAACGTAACACCAACCGTCGCCCACAGGACG | |
| Capture | GCAATTGGTTCGGTTGTACCTGGATGAACTCAACTGGATTCACCAAAG | SEQ ID NO: 3 |
| probe C | TGTGCGGAGCGCCCCCTTGTGTCATCGGAGGGGTGGGCAACA | |
| Capture | TGTCCACCACACAGTGGCAGGTCCTTCCGTGTTCTTTCACGACCCTGC | SEQ ID NO: 4 |
| probe D | CAGCCTTGTCCACCGGCCTCATCCACCTCCACCAGAACATTG | |
| Capture | ACCATGTTTCCCCCACGCACTACGTGCCGGAGAGCGATGCAGCCGCCC | SEQ ID NO: 5 |
| probe E | GCGTCACTGCCATACTCAGCAGCCTCACTGTAACCCAGCTCC | |
| Capture | GCTCCGGTTCCTGGCTAAGGGACATCTGGGACTGGATATGCGAGGTGC | SEQ ID NO: 6 |
| probe F | TGAGCGACTTTAAGACCTGGCTGAAAGCCAAGCTCATGCCAC | |
| Capture | TTATAACATCATGCTCCTCCAACGTGTCAGTCGCCCACGACGGCGCTG | SEQ ID NO: 7 |
| probe G | GAAAGAGGGTCTACTACCTTACCCGTGACCCTACAACCCCCC | |
| Capture | CCTGGCTAGGCAACATAATCATGTTTGCCCCCACACTGTGGGCGAGGA | SEQ ID NO: 8 |
| probe H | TGATACTGATGACCCATTTCTTTAGCGTCCTCATAGCCAGGG | |
| | qPCR primer set | |
| HCV-A- | CGGAATTGCCAGGACGAC | SEQ ID NO: 9 |
| upper | | |
| HCV-A- | GGATTCGTGCTCATGGTGC | SEQ ID NO: 10 |
| lower | | |
| HCV-B- | CTGCTAGCCGAGTAGTGTTGG | SEQ ID NO: 11 |
| upper | | |
| HCV-B- | GGAACTTGACGTCCTGTGG | SEQ ID NO: 12 |
| lower | | |
| HCV-C- | GGTATATTGCTTCACTCCCAGC | SEQ ID NO: 13 |
| upper | | |
| HCV-C- | CATCCAGGTACAACCGAACC | SEQ ID NO: 14 |
| lower | | |
| HCV-D- | GTCAGGATGTACGTGGGAGG | SEQ ID NO: 15 |
| upper | | |
| HCV-D- | CGTGAAAGAACACGGAAGG | SEQ ID NO: 16 |
| lower | | |
| HCV-E- | CAGCCTCACTGTAACCCAGC | SEQ ID NO: 17 |
| upper | | |
| HCV-E- | ACACAAAGGGAATCCCAGG | SEQ ID NO: 18 |
| lower | | |
| HCV-F- | TAAGGGACATCTGGGACTGG | SEQ ID NO: 19 |
| upper | | |
| HCV-F- | GTCCAGTGATCTCAGCTCCAC | SEQ ID NO: 20 |
| lower | | |
| HCV-G- | TCCTCCAACGTGTCAGTCG | SEQ ID NO: 21 |
| upper | | |
| HCV-G- | GGTCATCAGTATCATCCTCGC | SEQ ID NO: 22 |
| lower | | |
| HCV-H- | GGAGACAGCAAGACACACTCC | SEQ ID NO: 23 |
| upper | | |
| HCV-H- | CTATGGAGTAGCAGGCTCCG | SEQ ID NO: 24 |
| lower | | |

### Example 7—Capturing a Synthesized HCV Gene Fragment

Preparation of a Synthetic HCV Gene Fragment

A 270-bp long dsDNA of HCV sequence (gBlock gene fragment; SEQ ID NO: 25; below) was synthesized by IDT and ligated with Illumina adaptors to use as target fragments (containing on-target region A and B) in experiments for determining limit of detection and capture efficiency. In parallel, a library from genomic human DNA was also constructed. The target fragments were serially diluted 10 times from 10⁶ to 10 copies into 100 ng of the human DNA library. This simulated a DNA mixture extracted from human cells infected with relatively small amounts of HCV.

(SEQ ID NO: 25)
CCGGTGAGTACACCGGAATTGCCAGGACGACCGGGTCCTTTCTTGGATAA
ACCCGCTCAATGCCTGGAGATTTGGGCGTGCCCCCGCAAGACTGCTAGCC
GAGTAGTGTTGGGTCGCGAAAGGCCTTGTGGTACTGCCTGATAGGGTGCT
TGCGAGTGCCCCGGGAGGTCTCGTAGACCGTGCACCATGAGCACGAATCC
TAAACCTCAAAGAAAAACCAAACGTAACACCAACCGTCGCCCACAGGACG
TCAAGTTCCCGGGTGGCGGT

Target Sequence Capture and Enrichment with DNA Probe-Clicked Silica-Coated Iron Oxide Nanoparticles

The hepatitis C virus (HCV) is a pathogen that causes hepatitis and cirrhosis. During the initial stages of infection, most patients are asymptomatic, but approximately half of infected subjects will develop chronic infection. The disease often remains undiagnosed until serious liver damage has already occurred. To evaluate the efficiency of capture compounds disclosed herein in capturing and enriching of low abundance HCV targets, different sets of nanoparticles with probes spanning two distinct regions of the HCV genome (arbitrarily named A and B) were prepared as described in Example 6. The targets were synthetic DNA representing these genome regions, which are referenced herein as gblocks and were incorporated into Illumina TruSeq libraries.

Capture experiments using capture compounds of Example 6 were carried out to determine the limit of detection and to compare against qPCR results without capture (Table 3). DNA library (100 ng) was mixed with blocking reagents (2.5 μg of human cot-1 DNA, 2.5 μg salmon sperm DNA, and 300 pmol blocking oligos complement to library adaptor sequences) and denatured at 95° C. for 5 min, then maintained at 65° C. Hybridization buffer (containing 10× SSPE, 10× Denhardt's solution, 10 mM EDTA, 0.2% SDS and 20U RNase block) was freshly prepared and mixed with the DNA probe-clicked silica-coated iron oxide nanoparticles (20 μL stock solution, storage solution was removed before use), then pre-warmed to 65° C. and transferred to DNA library mix to make hybridization mix. The final hybridization mix (30 μL) was incubated at 65° C. for 24 h. Supernatant was removed and DNA probe-clicked silica-coated iron oxide nanoparticles (Example 6) were washed 3 times with wash buffer (0.1× SSC, 0.1% SDS) at 65° C. for 10 min each. Wash buffer was removed and the DNA probe-clicked silica-coated iron oxide nanoparticles were re-suspended with 30 μL ultra-pure water. On-beads PCR was performed using KAPA HiFi PCR kit (KAPA Biosystems) with following program: 98° C. 10 min; 98° C. 20 s, 58° C. 15 s, 72° C. 30 s, 35 cycles; 72v 10min; 4° C. indefinitely. The PCR product was purified by Qiaquick PCR Purification kit (Qiagen).

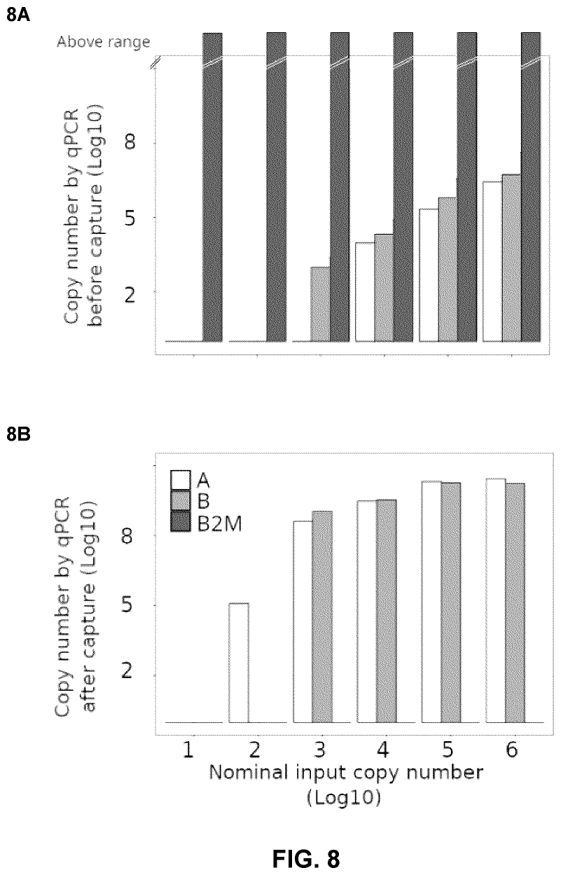

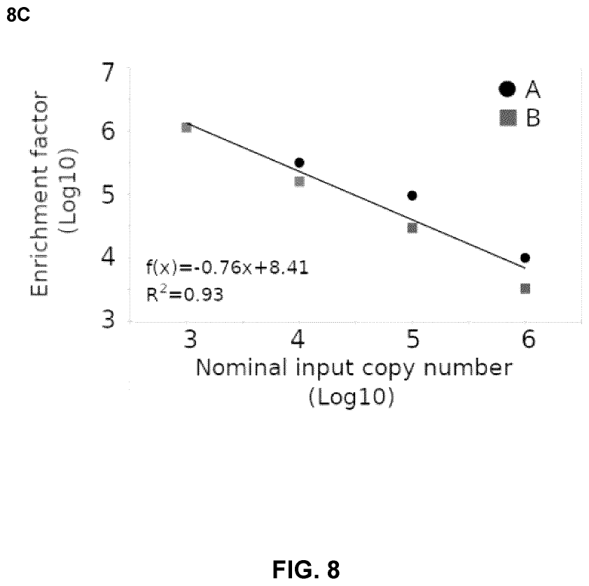

Amplification efficiency for the target sequence, assessed by real-time qPCR, decreased by adding a gDNA library background to the target fragment library. HCV gblocks could only be consistently detected in the dilutions containing 10⁴ or 10³ molecules from gblocks A and B, respectively (FIG. 8A). Recovery of these gblocks was substantially enriched (FIG. 8B and FIG. 8C) after capture. By using the capture compounds and re-amplification, a stable target signal (copy number >10⁸ per 100 ng) was obtained from experiments with 1000 copies of target per 100 ng or above, and the fluorescence signal from qPCR approached saturation at higher ends. Notably, when fluorescence signals approach the saturation point of qPCR detectors (e.g. 10⁹ or 10¹⁰ copy number), quantification often becomes imprecise. However, even as the saturation limit is approached, the relationship between the measured and actual signal is monotonic. So, if one signal is larger than another, it will remain larger when qPCR measurements are compared. Moreover, the actual ratio of these two signals will be larger than what is measured by qPCR. As such, results herein should be understood as possibly evenbetter than measured. The limit of detection after capture was 100 copies per 100 ng (1 copy per ng), with unstable results at lower dilutions corresponding to 10 copies of target fragment per 100 ng.

The housekeeping gene B2M was used to estimate the level of non-specific background with/without capture, and it was observed that a B2M signal with more than 10⁶ per 10Ong before capture (FIG. 8A) cannot be detected after capture (FIG. 8B) and re-amplification. This shows an enormous reduction in off-target effects and indicates that the capture compound system has very limited non-specific background DNA binding.

TABLE 3
Synthesized HCV Gene Fragment Capture

| Real-time | | | | | | |
|---|---|---|---|---|---|---|
| qPCR | | | | | | |
| (absolute | Without capture | | | After capture and re-amplification | | |
| quantification) | Measured copy number per 100 ng | | | Measured copy number per 100 ng | | |
| Primer set | A | B | B2M | A | B | B2M |
| 10\hat{ }6 gBlock in | 2.73E+06 | 5.30E+06 | >10\hat{ }6 | 2.69E+10 | 1.72E+10 | Undetermined |
| 100 ng gDNA | | | | | | |
| background | | | | | | |
| 10\hat{ }5 gBlock in | 2.16E+05 | 6.18E+05 | >10\hat{ }6 | 2.05E+10 | 1.80E+10 | Undetermined |
| 100 ng gDNA | | | | | | |
| background | | | | | | |
| 10\hat{ }4 gBlock in | 9.58E+03 | 2.11E+04 | >10\hat{ }6 | 3.00E+09 | 3.34E+09 | Undetermined |
| 100 ng gDNA | | | | | | |
| background | | | | | | |
| 10\hat{ }3 gBlock in | non- | 981 | >10\hat{ }6 | 4.09E+08 | 1.10E+09 | Undetermined |
| 100 ng gDNA | specific | | | | | |
| background | | | | | | |
| 10\hat{ }2 gBlock in | non- | Undetermined | >10\hat{ }6 | 1.24E+05 | Undetermined | Undetermined |
| 100 ng gDNA | specific | | | | | |
| background | | | | | | |
| 10\hat{ }1 gBlock in | non- | Undetermined | >10\hat{ }6 | Undetermined | Undetermined | Undetermined |
| 100 ng gDNA | specific | | | | | |
| background | | | | | | |

### Example 8—Target Sequence Enrichment Comparison with Streptavidin Beads

Capture compounds of Example 6 were prepared. The probes from 8 different locations of the HCV genome were chosen for a direct comparison between the capture protocols with DNA probe-clicked silica-coated iron oxide nanoparticles and streptavidin beads using the library built from a sample collected from three time-points of a patient with HCV positive by ELISA. The locations of the HCV genome chosen included some of the 5′ untranslated regions (UTRs) and regions encoding the core (coat) protein, the envelope glycoprotein E2, and three non-structural proteins associated with viral replication (NS4B, NS5A and NS5B). NGS libraries were constructed from an HCV-positive patient (PT339-1).

RNA extraction, reverse transcription, and library construction: Total RNA was extracted with a QIAamp viral RNA kit (Qiagen) from 1 mL plasma and cleaned using an RNeasy Mini kit with RNase-free DNase set (Qiagen). The RNA materials were reverse transcribed into cDNA using Superscript II Reverse Transcriptase (Thermo Fisher scientific) using random primers according to a standard protocol. HCV copy numbers were subsequently measured with TaqMan qPCR. Second strand cDNA was synthesized with reaction mix (Tris, pH 7.8; 50 mM MgCl₂; dNTP 10 mM; DTT 0.1 M; RNase H 2U/μL; DNA Polymerase I 10U/ μL) at 16° C. for 2.5 h. Double-stranded (ds) cDNA fragments were cleaned using the Qiaquick PCR Purification kit (Qiagen) and eluted in 40 μL ultrapure water (Gibco). Double-strand cDNA was sheared with Covaris-S2 to average size of 400 bp, followed by library construction with NEBNext Ultra II DNA library prep kit (NEB). Libraries were built from patient plasma samples with HCV copy number ranging from 100 or less to more than 10⁶ copies per mL plasma.

Target sequence enrichment by streptavidin beads: A pre-capture library mix (final volume 9 μL) was prepared by mixing 100 ng DNA library with blocking reagents and denaturing at 95° C. for 5 min, then maintained at 65° C. The capture probe mix was prepared by mixing 500 ng biotin-labeled probe set (pooled the eight 5′-biotin-labeled probes at equal concentration) with 20U RNase Block and ultra-pure water to final volume of 7 μL, then pre-warmed to 65° C. for at least 2 min. Hybridization buffer (containing 10× SSPE, 10× Denhardt's solution, 10 mM EDTA, 0.2% SDS and 20U RNase block) was freshly prepared and pre-warmed at 65° C. for at least 5 min. 13 μL hybridization buffer and 7 μL capture probe mix were rapidly added to pre-capture library mix to make the hybridization mix with final volume of 29 μL and maintain at 65° C. for 24 h. 50 μL of Dynabeads MyOne streptavidin T1 beads was washed twice with 200 μL Binding buffer and re-suspended with 200 μL binding buffer. The hybridization mix was directly transferred to beads solution and incubated on a rotator at room temperature for 30 min, then the supernatant was discarded. The beads were washed once with 500 μL wash buffer I (1× SSC, 0.1% SDS) at room temperature for 15 min, then washed with 500 μL wash buffer II (0.1× SSC, 0.1% SDS) at 65° C. for 10 min and repeated three times. After removing the wash buffer, the beads were re-suspended with 30 μL ultra-pure water. The on-beads PCR was performed using KAPA HiFi PCR kit (KAPA Biosystems) with following program: 98° C. 10 min; 98° C. 20 s, 58° C. 15 s, 72° C. 30 s, 35 cycles; 72° C. 10 min; 4° C. indefinitely. The PCR product was purified by Qiaquick PCR Purification kit (Qiagen) before performing qPCR.

Post-capture amplification by on-beads qPCR: Quantitative polymerase chain reaction (qPCR) assays with SYBR Green PCR Mastermix (Thermo Fisher scientific) were developed to quantify the copy numbers of the target sequences after post-capture on-beads PCR. 8 pairs of primers were designed for post-capture qPCR assay; for each pair, one primer was designed in the on-target region and the other one in the flanking regions to ensure a PCR product length within range of 150-200 bp and to avoid amplifying probe sequences. 2 μL of purified on-beads PCR product was used for each qPCR reaction with final volume of 25 μL, and for each pair of primers, the amplification reactions were carried out in duplicate using ABI 3700 real time PCR system (ABI). The reaction started at 95° C. for 10 min and proceeded with 40 cycles of 95° C. for 15 s and 60° C. for 1 min. A final dissociation step was performed to obtain the melting curve. The copy number of target sequences was calculated based on the standard curve that was generated with serially diluted plasmid with HCV genomic sequence (from 10⁶ to 10 copies per μL).

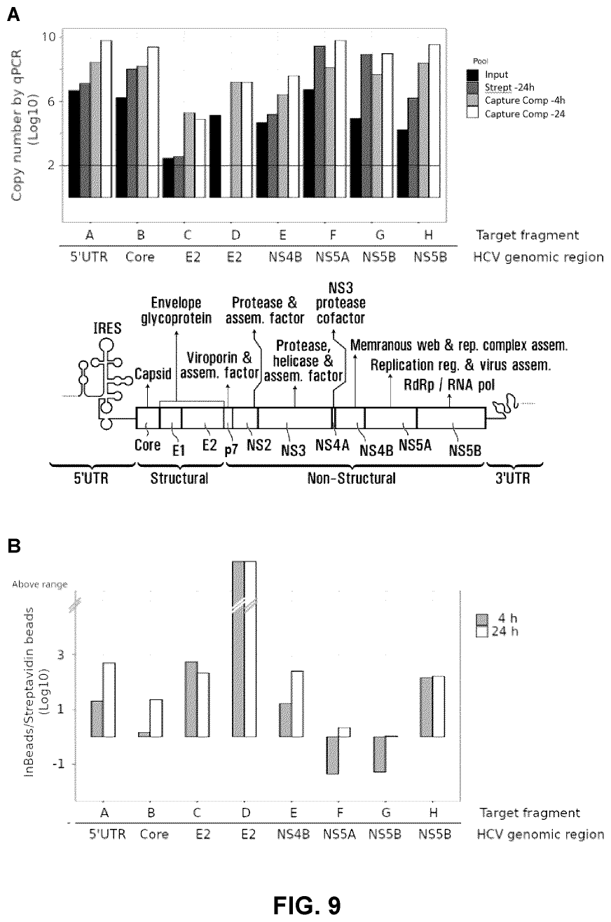

With 24 hours hybridization, results showed that the DNA probe-clicked silica-coated iron oxide nanoparticles were on average 165× more effective than Dynabeads, particular over regions A, C, E and H, corresponding to 5′UTR, E2, NS4B and NSSB, respectively (FIGS. 9A and 9B). Moreover, stable signals from experiments with the DNA probe-clicked silica-coated iron oxide nanoparticles yielded 100-1000 times more copies of the target sequences compared to using protocol with streptavidin beads, especially in the on-target region C, D and E that located in HCV E2 and NS4B genes (see Table 4). At 24 h of hybridization, the DNA probe-clicked silica-coated iron oxide nanoparticles outperformed streptavidin in all regions, especially in region D where streptavidin failed outright. The performance of the capture protocol with the DNA probe-clicked silica-coated iron oxide nanoparticles was found to be very stable with signal of target more than 10⁵ copies per μL.

To reduce the overall turnaround time of the capture assay, a rapid capture protocol with 4-hour hybridization instead of 24-hour hybridization was developed. Even at the reduced 4 h hybridization, the DNA probe-clicked silica-coated iron oxide nanoparticles outperformed streptavidin in all but two regions (FIG. 9B). The target signal was still very stable in repeated experiments with average copy number more than 10⁵ per assay, though the overall capture efficiency was lower than with a 24-hour hybridization. However, on average, the DNA probe-clicked silica-coated iron oxide nanoparticles performed 103× better than Dynabeads.

TABLE 4
DNA Probe-Clicked Silica-coated Iron Oxide Nanoparticles
vs. Streptavidin Beads Capture Protocols

| | | | | DNA | DNA |
|---|---|---|---|---|---|
| | | | | probe-clicked | probe-clicked |
| | | Copy | | silica-coated | silica-coated |
| | | number of | Streptavidin | iron oxide | iron oxide |
| | | HCV | beads- | nanoparticles - | nanoparticles - |
| | | fragments | 24 h | 24 h | 4 h |
| | On-target | per ml | after capture | after capture | after capture |
| Gene | regions | plasma | (copies per μL) | (copies per μL) | (copies per μL) |
| 5′ UTR | A | 4.82E+06 | 1.26E+07 | 6.16E+09 | 2.59E+08 |
| core | B | 1.74E+06 | 1.07E+08 | 2.47E+09 | 1.56E+08 |
| E2 | C | 283 | 352 | 7.72E+04 | 1.89E+05 |
| | D | 1.38E+05 | <100 (non-specific | 1.54E+07 | 1.50E+07 |
| | | | product) | | |
| NS4B | E | 4.72E+04 | 1.58E+05 | 4.02E+07 | 2.62E+06 |
| NS5A | F | 5.49E+06 | 2.87E+09 | 6.23E+09 | 1.27E+08 |
| NS5B | G | 8.36E+04 | 8.80E+08 | 9.48E+08 | 4.57E+07 |
| | H | 1.72E+04 | 1.64E+06 | 3.67E+09 | 2.42E+08 |

### Example 9—HCV Gene Panel from Patient Samples

Preparation of Libraries from Patient Samples

The following plasma samples were collected: two time-points of a patient (Pt804) with HCC (Hepatocellular carcinoma) and HCV (Hepatitis C virus) positive by ELISA; three time-points of a patient (Pt339) without HCC and HCV positive by ELISA; and from a control patient (Pt555) without HCC or HCV. Total RNA was extracted with a QIAamp viral RNA kit (Qiagen) from 1 mL plasma and cleaned using an RNeasy Mini kit with RNase-free DNase set (Qiagen). The RNA materials were reverse transcribed into cDNA using Superscript II Reverse Transcriptase (Thermo Fisher scientific) using random primers according to a standard protocol. HCV copy numbers were subsequently measured with TaqMan qPCR. Second strand cDNA was synthesized with reaction mix (Tris, pH 7.8; 50 mM MgCl₂; dNTP 10 mM; DTT 0.1 M; RNase H 2U/μL; DNA Polymerase I 10U/ μL) at 16° C. for 2.5 h. Double-stranded (ds) cDNA fragments were cleaned using the Qiaquick PCR Purification kit (Qiagen) and eluted in 40 μL ultrapure water (Gibco). Double-strand cDNA was sheared with Covaris-S2 to average size of 400 bp, followed by library construction with NEBNext Ultra II DNA library prep kit (NEB). Libraries were built from patient plasma samples with HCV copy number.

Fast Capture Protocol

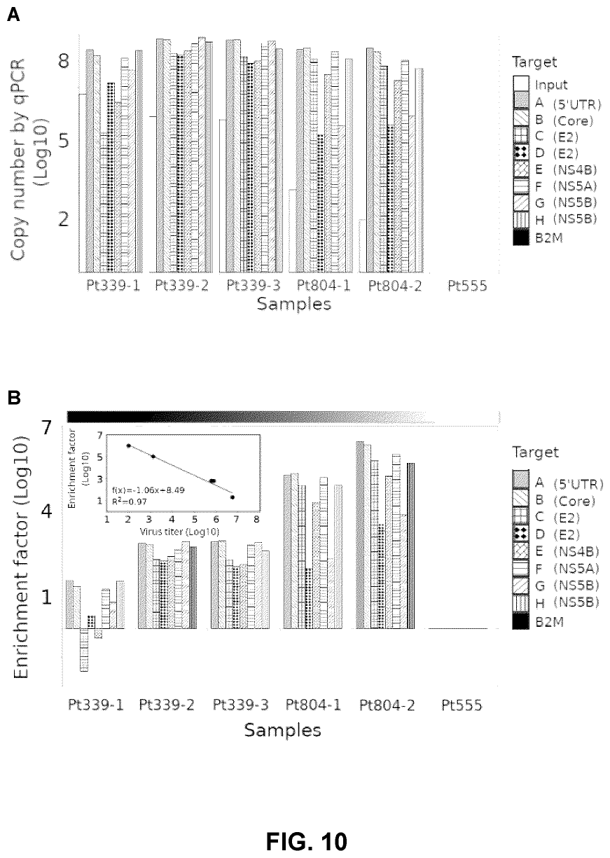

The performance of the DNA probe-clicked silica-coated iron oxide nanoparticles on plasma samples from HCV patients, with viral titers ranging from below 100 copies to over 106 copies per mL was studied. The 4-hour hybridization protocol was performed and a consistently stable signal from all 8 HCV on-target regions was observed, even in the sample with HCV copies below the limit of detection of qPCR assay without capture but for which the virus was known to be present because the patient later suffered a relapse (Pt804-2). A signal from the negative control sample (Pt555) or the housekeeping gene B2M was not observed using the capture protocol with the DNA probe-clicked silica-coated iron oxide nanoparticles (Table 5 and FIG. 10). For example, sample Pt339-1 had a high viral titer (>5.5×10⁶) and the enrichment factor averaged 19× (FIG. 10B). But sample Pt804-2, where the virus was below the detection limit of qPCR, showed better than 9×10⁵× enrichment factor (FIG. 10B, inset). This demonstrates that the DNA probe-clicked silica-coated iron oxide nanoparticles capture protocol has high sensitivity and specificity, and limited non-specific binding, making a gene panel based on the DNA probe-clicked silica-coated iron oxide nanoparticles system suitable for low abundant target enrichment and rapid diagnostic assays.

Altogether, these data show that DNA probe-clicked silica-coated iron oxide nanoparticles outperform streptavidin-conjugated beads in capture of low abundance sequences from clinical samples. Moreover, given the high efficiency of DNA probe-clicked silica-coated iron oxide nanoparticles, it is possible to shorten the hybridization step to 4 h (down from 24 h), reducing turnaround times with just a minor loss in performance

TABLE 5

| | HCV copy | | | | | | | | | |
|---|---|---|---|---|---|---|---|---|---|---|
| | number | Copy number per μL after capture with DNA probe-clicked | | | | | | | | |
| | per ml | silica-coated iron oxide nanoparticles (4 hours incubation) | | | | | | | | |
| Pt ID | plasma | A | B | C | D | E | F | G | H | B2M |
| pt 339-1 | 5,567,875 | 2.59E+08 | 1.56E+08 | 1.89E+05 | 1.50E+07 | 2.62E+06 | 1.27E+08 | 4.57E+07 | 2.42E+08 | 0 |
| pt 339-2 | 773,345 | 6.72E+08 | 6.24E+08 | 1.90E+08 | 1.63E+08 | 2.38E+08 | 4.31E+08 | 7.75E+08 | 5.04E+08 | 0 |
| pt 339-3 | 599,427 | 6.05E+08 | 6.25E+08 | 1.41E+08 | 8.19E+07 | 9.77E+07 | 4.63E+08 | 5.68E+08 | 2.85E+08 | 0 |
| pt 804-1 | 1342 | 2.62E+08 | 3.08E+08 | 1.17E+08 | 1.57E+05 | 3.05E+07 | 2.25E+08 | 3.55E+05 | 1.19E+08 | 0 |
| pt 804-2 | <100 | 2.98E+08 | 2.20E+08 | 6.16E+07 | 3.79E+05 | 1.81E+07 | 1.05E+08 | 8.46E+05 | 5.25E+07 | 0 |
| pt 555 | 0 | 0 | 0 | 0 | 0 | 0 | 0 | 0 | 0 | 0 |
| (NC) | | | | | | | | | | |

For the sake of brevity, only certain ranges are explicitly disclosed herein. However, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

### REFERENCES

1. Saunders, N. A., Application of nanomaterials to arrays for infectious disease diagnosis. Nanomedicine 2011, 6 (2), 271-280.
2. Dederich, D. A.; Okwuonu, G.; Garner, T.; Denn, A.; Sutton, A.; Escotto, M.; Martindale, A.; Delgado, 0.; Muzny, D. M.; Gibbs, R. A., Glass bead purification of plasmid template DNA for high throughput sequencing of mammalian genomes. Nucleic acids research 2002, 30 (7), e32-e32.
3. Bruce, I. J.; Sen, T., Surface modification of magnetic nanoparticles with alkoxysilanes and their application in magnetic bioseparations. Langmuir 2005, 21 (15), 7029-7035.
4. Levicky, R.; Horgan, A., Physicochemical perspectives on DNA microarray and biosensor technologies. Trends in Biotechnology 2005, 23 (3), 143-149.
5. Tan, S. C.; Yiap, B. C., DNA, RNA, and protein extraction: the past and the present. BioMed Research International 2009, Article ID 574398.
6. Ji Hyun Min, M.-K. W., Ha Young Yoon, Jin Woo Jang, Jun Hua Wu, Chae-Seung Lim, Young Keun Kim, Isolation of DNA using magnetic nanoparticles coated with dimercaptosuccinic acid. Analytical Biochemistry 2014, 447, 114-118.
7. Issa, B.; Obaidat, I. M.; Albiss, B. A.; Haik, Y., Magnetic nanoparticles: surface effects and properties related to biomedicine applications. International journal of molecular sciences 2013, 14 (11), 21266-21305.
8. Erathodiyil, N.; Ying, J. Y., Functionalization of Inorganic Nanoparticles for Bioimaging Applications. Accounts of Chemical Research 2011, 44 (10), 925-935.
9. Berensmeier, S., Magnetic particles for the separation and purification of nucleic acids. Applied microbiology and biotechnology 2006, 73 (3), 495-504.
10. Corchero, J. L.; Villaverde, A., Biomedical applications of distally controlled magnetic nanoparticles. Trends in biotechnology 2009, 27 (8), 468-476.
11. Saiyed, Z.; Parasramka, M.; Telang, S.; Ramchand, C., Extraction of DNA from agarose gel using magnetic nanoparticles (magnetite or Fe 3 O 4). Analytical biochemistry 2007, 363 (2), 288-290.
12. Paul, A.; Avci-Adali, M.; Ziemer, G.; Wendel, H. P., Streptavidin-coated magnetic beads for DNA strand separation implicate a multitude of problems during cell-SELEX. Oligonucleotides 2009, 19 (3), 243-254.
13. Oktem, H. A.; Bayramoglu, G.; Ozalp, V. C.; Arica, M. Y., Single-Step Purification of Recombinant Thermus aquaticus DNA Polymerase Using DNA-Aptamer Immobilized Novel Affinity Magnetic Beads. Biotechnology progress 2007, 23 (1), 146-154.
14. Kang, K.; Choi, J.; Nam, J. H.; Lee, S. C.; Kim, K. J.; Lee, S.-W.; Chang, J. H., Preparation and characterization of chemically functionalized silica-coated magnetic nanoparticles as a DNA separator. The Journal of Physical Chemistry B 2008, 113 (2), 536-543.
15. Rittich, B.; Španová, A.; Horák, D.; Beneš, M.; Klesnilová, L.; Petrová K.; Rybnikář, A., Isolation of microbial DNA by newly designed magnetic particles. Colloids and Surfaces B: Biointerfaces 2006, 52 (2), 143-148.
16. Shao, D.; Xia, A.; Hu, J.; Wang, C.; Yu, W., Monodispersed magnetite/silica composite microspheres: preparation and application for plasmid DNA purification. Colloids and Surfaces A: Physicochemical and Engineering Aspects 2008, 322 (1), 61-65.
17. Gijs, M. A. M.; Lacharme, F.; Lehmann, U., Microfluidic Applications of Magnetic Particles for Biological Analysis and Catalysis. Chemical Reviews 2010, 110 (3), 1518-1563.
18. Thomson, D. A. C.; Tee, E. H. L.; Tran, N. T. D.; Monteiro, M. J.; Cooper, M. A., Oligonucleotide and Polymer Functionalized Nanoparticles for Amplification-Free Detection of DNA. Biomacromolecules 2012, 13 (6), 1981-1989.
19. Downs, M. E.; Kobayashi, S.; Karube, I., New DNA technology and the DNA biosensor. Analytical letters 1987, 20 (12), 1897-1927.
20. Mahmoudi, M.; Sant, S.; Wang, B.; Laurent, S.; Sen, T., Superparamagnetic iron oxide nanoparticles (SPIONs): development, surface modification and applications in chemotherapy. Advanced drug delivery reviews 2011, 63 (1), 24-46.
21. Wu, W.; Wu, Z.; Yu, T.; Jiang, C.; Kim, W.-S., Recent progress on magnetic iron oxide nanoparticles: synthesis, surface functional strategies and biomedical applications. Science and Technology of Advanced Materials 2016.
22. Gong, P.; Grainger, D. W., Comparison of DNA immobilization efficiency on new and regenerated commercial amine-reactive polymer microarray surfaces. Surface science 2004, 570 (1), 67-77.
23. Shan, Z.; Jiang, Y.; Guo, M.; Bennett, J. C.; Li, X.; Tian, H.; Oakes, K.; Zhang, X.; Zhou, Y.; Huang, Q.; Chen, H., Promoting DNA loading on magnetic nanoparticles using a DNA condensation strategy. Colloids and Surfaces B: Biointerfaces 2015, 125, 247-254.
24. Vessman, J.; Stefan, R. I.; van Staden, J. F.; Danzer, K.; Linder, W.; Burns, D. T.; Fajgelj, A.; Muller, H., Selectivity in Analytical Chemistry (IUPAC Recommendations 2001). Pure Applied Chemistry 2001, 73, 1381-1386.
25. Haque, M. M.; Peng, X. H., DNA-associated click chemistry. Science China-Chemistry 2014, 57 (2), 215-231.
26. Hong, V.; Presolski, S. I.; Ma, C.; Finn, M. G., Analysis and Optimization of Copper-Catalyzed Azide-Alkyne Cycloaddition for Bioconjugation. Angewandte Chemie-International Edition 2009, 48 (52), 9879-9883.
27. Li, N. W.; Binder, W. H., Click-chemistry for nanoparticle-modification. Journal of Materials Chemistry 2011, 21 (42), 16717-16734.
28. Breinbauer, R.; K\check{o}hn, M., Azide-Alkyne Coupling: A Powerful Reaction for Bioconjugate Chemistry. ChemBioChem 2003, 4 (11), 1147-1149.
29. Tian, Y.; Yu, B.; Li, X; Li, K., Facile solvothermal synthesis of monodisperse Fe₃O₄ nanocrystals with precise size control of one nanometre as potential MRI contrast agents. J. Mater. Chem. 2011, 21, 2476-2481.
30. Stöber, W.; Fink, A; Bohn, E., Controlled growth of monodisperse silica spheres in the micron size range. J. Colloid Interface Sci. 1968, 26, 62-69.
31. Schatz, A.; Hager, M.; Reiser, O., Cu(II)-Azabis(oxazoline)-Complexes Immobilized on Superparamagnetic Magnetite©Silica-Nanoparticles: A Highly Selective and Recyclable Catalyst for the Kinetic Resolution of 1,2-Diols. Adv. Funct. Mater. 2009, 19, 2109-2115.

1. A method for capturing a low abundant target biomolecule, the method comprising:
providing a capture compound comprising a silica-coated nanoparticle conjugated to one or more biomolecule probes; and
incubating the capture compound with a sample comprising the low abundant target biomolecule to capture the low abundant target biomolecule.

2. The method of claim 1, wherein the silica-coated nanoparticle is conjugated to the one or more biomolecule probes by a triazole linkage.

3. (canceled)

4. The method according to claim 1, wherein the low abundant target biomolecule is less than 5% of a total genomic background in the sample.

5-6. (canceled)

7. The method according to claim 1, which is for capturing a low abundant target nucleic acid of a viral genome, a bacterial genome, or a fungal genome; or a low abundant target nucleic acid that comprises a mutated or non-mutated nucleotide sequence of a nucleic acid of a genome.

8-12. (canceled)

13. The method according to claim 1, wherein the nanoparticle of the silica-coated nanoparticle is a magnetic nanoparticle.

14. (canceled)

15. The method according to claim 1, wherein the one or more biomolecule probes are oligonucleotide probes and form a dense monolayer on the silica.

16-19. (canceled)

20. The method according to claim 15, wherein the one or more oligonucleotide probes comprise a nucleotide sequence that has at least 70% complementarity to a low abundant target nucleic acid.

21-22. (canceled)

23. The method of according to claim 1, wherein, prior to incubating, the capture compound is unbound in the sample.

24-27. (canceled)

28. The method according to claim 13, further comprising isolating or separating the low abundant target biomolecule from the sample by magnetic attraction between a magnetic source and the capture compound.

29-30. (canceled)

31. The method according to claim 1, wherein the low abundant target biomolecule is present at less than 10⁶ copies/100 ng of DNA in the sample.

32. (canceled)

33. A method of enriching a low abundant target biomolecule in a sample, comprising:
providing an unbound capture compound comprising a silica-coated nanoparticle conjugated to one or more biomolecule probes;
incubating the capture compound with a sample comprising the low abundant target biomolecule; and
performing an amplification to enrich the low abundant target biomolecule in the sample.

34. The method of claim 33, wherein the silica-coated nanoparticle is conjugated to the one or more biomolecule probes by a triazole linkage and the nanoparticle of the silica-coated nanoparticle is a magnetic nanoparticle.

35. The method according to claim 33, wherein the amplification is a polymerase chain reaction, and the polymerase chain reaction is an on-beads polymerase chain reaction.

36. (canceled)

37. The method according to claim 35, further comprising performing a quantitative polymerase chain reaction (qPCR) on an amplification product of the on-beads PCR.

38. (canceled)

39. The method according to claim 33, which is for enriching a low abundant target nucleic acid of a viral genome, a bacterial genome, or a fungal genome; or a low abundant target nucleic acid comprising a mutated or non-mutated nucleotide sequence of a nucleic acid of a genome.

40-55. (canceled)

56. The method according to claim 33, wherein prior to enrichment the low abundant target biomolecule; is less than 5% of a total genomic background in the sample and/or is present at less than 10⁶ copies/100 ng of DNA in the sample.

57-58. (canceled)

59. A method for diagnosis or prognosis of a disease and/or disorder associated with an infectious agent or a mutated or non-mutated nucleotide sequence of a nucleic acid of a genome, the method comprising:
providing a sample; and
capturing a low abundant target biomolecule of an infectious agent and/or a low abundant target nucleic acid which comprises a mutated or non-mutated nucleotide sequence of a nucleic acid of a genome, by using a capture compound comprising a silica-coated nanoparticle conjugated to one or more biomolecule probes in a capture assay.

60. The method of claim 59, wherein the silica-coated nanoparticle is conjugated to the one or more biomolecule probes by a triazole linkage and the nanoparticle of the silica-coated nanoparticle is a magnetic nanoparticle.

61-63. (canceled)

64. The method according to claim 59, wherein the low abundant target biomolecule or low abundant target nucleic acid is present at less than 10⁶ copies/100 ng of DNA in the sample.

65-67. (canceled)

68. The method of claim 59, wherein for prognosis the method further comprises the step of:
detecting for a quantity of the low abundant target biomolecule and/or the low abundant target nucleic acid,
wherein either:
an elevated or reduced level of the low abundant target biomolecule and/or the low abundant target nucleic acid in the sample as compared to a predefined value is indicative of a poor prognosis or active disease state; or
an elevated or reduced level of the low abundant target biomolecule and/or the low abundant target nucleic acid in the sample as compared to an earlier sample is indicative of a poor prognosis or active disease state.

69-89. (canceled)


SEQUENCE LISTING (25 sequences)

SEQ ID NO: 1 - DNA, 90 bp, Artificial Sequence
  Synthetic Sequence
tttgggcgtgcccccgcaagactgctagccgagtagtgttgggtcgcgaaaggccttgtggtactgcctgatagggtgcttgcgagtgcc

SEQ ID NO: 2 - DNA, 90 bp, Artificial Sequence
  Synthetic Sequence
ccgggaggtctcgtagaccgtgcaccatgagcacgaatcctaaacctcaaagaaaaaccaaacgtaacaccaaccgtcgcccacaggacg

SEQ ID NO: 3 - DNA, 90 bp, Artificial Sequence
  Synthetic Sequence
gcaattggttcggttgtacctggatgaactcaactggattcaccaaagtgtgcggagcgcccccttgtgtcatcggaggggtgggcaaca

SEQ ID NO: 4 - DNA, 90 bp, Artificial Sequence
  Synthetic Sequence
tgtccaccacacagtggcaggtccttccgtgttctttcacgaccctgccagccttgtccaccggcctcatccacctccaccagaacattg

SEQ ID NO: 5 - DNA, 90 bp, Artificial Sequence
  Synthetic Sequence
accatgtttcccccacgcactacgtgccggagagcgatgcagccgcccgcgtcactgccatactcagcagcctcactgtaacccagctcc

SEQ ID NO: 6 - DNA, 90 bp, Artificial Sequence
  Synthetic Sequence
gctccggttcctggctaagggacatctgggactggatatgcgaggtgctgagcgactttaagacctggctgaaagccaagctcatgccac

SEQ ID NO: 7 - DNA, 90 bp, Artificial Sequence
  Synthetic Sequence
ttataacatcatgctcctccaacgtgtcagtcgcccacgacggcgctggaaagagggtctactaccttacccgtgaccctacaacccccc

SEQ ID NO: 8 - DNA, 90 bp, Artificial Seqeuence
  Synthetic Sequence
cctggctaggcaacataatcatgtttgcccccacactgtgggcgaggatgatactgatgacccatttctttagcgtcctcatagccaggg

SEQ ID NO: 9 - DNA, 18 bp, Artificial Sequence
  Synthetic Sequence
cggaattgccaggacgac

SEQ ID NO: 10 - DNA, 19 bp, Artificial Sequence
  Synthetic Sequence
ggattcgtgctcatggtgc

SEQ ID NO: 11 - DNA, 21 bp, Artificial Sequence
  Synthetic Sequence
ctgctagccgagtagtgttgg

SEQ ID NO: 12 - DNA, 19 bp, Artificial Sequence
  Synthetic Sequence
ggaacttgacgtcctgtgg

SEQ ID NO: 13 - DNA, 22 bp, Artificial Sequence
  Synthetic Sequence
ggtatattgcttcactcccagc

SEQ ID NO: 14 - DNA, 20 bp, Artificial Sequence
  Synthetic Sequence
catccaggtacaaccgaacc

SEQ ID NO: 15 - DNA, 20 bp, Artificial Sequence
  Synthetic Seqeunce
gtcaggatgtacgtgggagg

SEQ ID NO: 16 - DNA, 19 bp, Artificial Sequence
  Synthetic Sequence
cgtgaaagaacacggaagg

SEQ ID NO: 17 - DNA, 20 bp, Artificial Sequence
  Synthetic Sequence
cagcctcactgtaacccagc

SEQ ID NO: 18 - DNA, 19 bp, Artificial Sequence
  Synthetic Sequence
acacaaagggaatcccagg

SEQ ID NO: 19 - DNA, 20 bp, Artificial Sequence
  Synthetic Sequence
taagggacatctgggactgg

SEQ ID NO: 20 - DNA, 21 bp, Artificial Sequence
  Synthetic Sequence
gtccagtgatctcagctccac

SEQ ID NO: 21 - DNA, 19 bp, Artificial Sequence
  Synthetic Sequence
tcctccaacgtgtcagtcg

SEQ ID NO: 22 - DNA, 21 bp, Artificial Sequence
  Synthetic Sequence
ggtcatcagtatcatcctcgc

SEQ ID NO: 23 - DNA, 21 bp, Artificial Sequence
  Synthetic Sequence
ggagacagcaagacacactcc

SEQ ID NO: 24 - DNA, 20 bp, Artificial Sequence
  Synthetic Sequence
ctatggagtagcaggctccg

SEQ ID NO: 25 - DNA, 270 bp, Artificial Sequence
  Synthetic Sequence
ccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggt